(19) United States
(12) Patent Application Publication
(10) Pub. No.: US 2013/0253518 A1
(43) Pub. Date: Sep. 26, 2013

(54) SYSTEM AND METHOD FOR CREATING A BORE AND IMPLANTING A BONE SCREW IN A VERTEBRA

(71) Applicant: SPARTEK MEDICAL, INC., Concord, CA (US)

(72) Inventors: Steven T. Mitchell, Pleasant Hill, CA (US); Charles J. Winslow, Lafayette, CA (US); James F. Zucherman, San Francisco, CA (US); Ken Y. Hsu, San Francisco, CA (US)

(73) Assignee: SPARTEK MEDICAL, INC., Concord, CA (US)

(21) Appl. No.: 13/735,215

(22) Filed: Jan. 7, 2013

Related U.S. Application Data
(63) Continuation-in-part of application No. 13/434,652, filed on Mar. 29, 2012.
Application No. 13/434,652 is a continuation-in-part of application No. 13/434,674, filed on Mar. 29, 2012.
(60) Provisional application No. 61/615,639, filed on Mar. 26, 2012.
(60) Provisional application No. 61/725,771, filed on Nov. 13, 2012.

(51) Int. Cl. A61B 17/16

(52) U.S. Cl. CPC A61B 17/1671
USPC 606/80

(57) ABSTRACT

A system and method for implanting a bone screw in bone which includes the use of a bone cutting tool with expanding bone cutting blades that create a bore in the bone of a desired shape and size. Then a bone screw is introduced into the bore and bone cement is positioned between the bone screw and the bore to fix the position of the bone screw relative to the bone.

### CLAIM TO PRIORITY

This application claims the benefit of priority to U.S. Provisional Patent Application No. 61/725,771, filed Nov. 13, 2012, entitled “SYSTEM AND METHOD FOR IMPLANTING A BONE SCREW IN A VERTEBRA”; and

This application claims the benefit of priority to and is a continuation-in-part of:

U.S. patent application Ser. No. 13/434,652, filed Mar. 29, 2012, entitled “SYSTEM AND METHOD FOR SECURING AN IMPLANT TO A BONE CONTAINING BONE CEMENT”; and

U.S. patent application Ser. No. 13/434,674, filed Mar. 29, 2012, entitled “SYSTEM AND METHOD FOR SECURING AN IMPLANT TO A BONE CONTAINING BONE CEMENT”; and which claims the benefit of priority to:

U.S. Provisional Application No. 61/615,639, filed Mar. 26, 2012, entitled “SYSTEM AND METHOD FOR SECURING AN IMPLANT TO A BONE CONTAINING BONE CEMENT” which all of the above applications are herein incorporated by reference in their entirety.

### BACKGROUND OF INVENTION

Back pain is a significant clinical problem and the costs to treat it, both surgical and medical, are estimated to be over $2 billion per year. One method for treating a broad range of degenerative spinal disorders is spinal fusion. Implantable medical devices designed to fuse vertebrae of the spine have developed rapidly over the last decade. However, spinal fusion has several disadvantages including reduced range of motion and accelerated degenerative changes adjacent the fused vertebrae. Alternative devices and treatments have been developed for treating degenerative spinal disorders while preserving motion. These devices and treatments offer the possibility of treating degenerative spinal disorders without the disadvantages of spinal fusion.

Devices for treating the spine, including those used in spinal fusion and spinal stabilization with motion preservation, are typically secured to the spine using screws which penetrate the bone. Such screws are designed to engage the structure of the bone. However, such screws are poorly adapted for use in bones which have been previously treated with bone cement. Consequently, there is a need for new and improved devices and methods for securing spinal implants to vertebrae that have previously been treated with bone cement.

### SUMMARY OF INVENTION

Systems and methods of the embodiments of the present invention include a bone cutting tool that can be used to create a bore in a vertebral body in order to implant a bone screw with the aid of bone cement. Embodiments of the bone cutting tool of the invention include at least an outer bone cutting blade and an inner rod, preferably, an outer tube with first and second bone cutting blades and an inner rod. Movement of the inner rod causes the first and second bone cutting blades to expand. Rotating the tool causes bone to be cut and a bore in which the tool is placed to expand. Continued expansion of the bone cutting blades and rotation of the tool cause the bore to expand. The expanded bore can be cylindrical due to a cylindrical shape of the bone cutting blades. Once the bore has a desired size, the bone cutting blades can be retracted and the tool removed from the bore.

Embodiments of the invention use the bone cutting tool to create a bore of the desired size. After the bone cutting tool is removed, a bone screw is inserted and bone cement is used to affix the bone screw into the vertebra. The bone cement may be applied between the bone screw and the bore. Alternatively and/or additionally, the bone cement may be applied through a bore and channels in the bone screw and exit through a port in the bone screw to fill the space between the bone screw and the bore in the vertebra.

The present invention includes a bone anchor system and methods that can secure a spinal implant to a vertebra that has previously been treated with bone cement. Embodiments of the invention include polyaxial bone anchors; dynamic bone anchors; bone screws adapted to engage bone and hardened bone cement in a bone, and methods of implantation.

An aspect of embodiments of the invention is the ability of the bone anchor system to engage both bone and hardened bone cement with a single anchor. Another aspect of embodiments of the invention is the ability to provide a kit of versatile components suitable for particular bones of the patient and which may be customized to the anatomy and needs of a particular patient and procedure. Another aspect of the invention is to facilitate the process of implantation of the bone anchor and minimize disruption of the bone and hardened bone cement during implantation.

Thus, the present invention provides new and improved systems, devices and methods for treating degenerative spinal disorders by providing and implanting a bone anchor system adapted to engage bone and hardened bone cement in a bone. These and other objects, features and advantages of the invention will be apparent from the drawings and detailed description which follow.

### BRIEF DESCRIPTION OF THE DRAWINGS

FIGS. 1A and 1B are front and back perspective views of a bone anchor according to an embodiment of the present invention.

FIG. 1C is a sectional view of the bone anchor of FIGS. 1A and 1B.

FIGS. 1D, 1E, and 1F are enlargements of portions of FIG. 1C.

FIGS. 2A-2D illustrate steps in the implantation of the bone anchor of FIGS. 1A and 1B into a vertebra according to an embodiment of the invention.

FIGS. 2E-2I illustrate steps in the implantation of a bone anchor into a vertebra according to alternative embodiments of the invention.

FIGS. 3A-3H show illustrative views of alternative bone anchors according to embodiments of the present invention.

FIGS. 4A-4F illustrative views of alternative bone anchor cross-sections according to embodiments of the present invention.

FIGS. 5A-5D show illustrative views of alternative tips of bone anchors according to embodiments of the present invention

FIGS. 6A-6F show illustrative views of bone anchor heads which can be combined with the shaft of the bone anchors shown in FIGS. 1A-5D.

FIGS. 7A-7C show views of a dynamic bone anchor head in combination with the shaft of the bone anchor shown in FIGS. 1A-1F.

FIGS. 8A-8D show illustrative views of alternative bone anchors which can be combined with the shaft of the bone anchors shown in FIGS. 1A-5D.

FIGS. 9A-9F show illustrative views of alternative bone anchors having heated tips which can be combined with the shaft of the bone anchors shown in FIGS. 1A-5D.

FIGS. 10A-10B show perspective views of a bone cutting tool in a non-expanded mode and an expanded mode of an embodiment of the invention.

FIG. 10C shows a perspective view of the first and second cutting blade of the embodiment of the invention.

FIGS. 11A-11B show side views of a bone cutting tool in a non-expanded mode and an expanded mode of an embodiment of the invention.

FIG. 12A shows a cross-sectioned view of the bone cutting tool of an embodiment of the invention as depicted in FIG. 10A.

FIG. 12B shows a perspective view of the proximal end of the bone cutting tool of an embodiment of the invention.

FIG. 12C shows a close-up side view of the first and second cutting blade of an embodiment of the invention in an unexpanded configuration.

FIG. 12D shows a close-up of an alternative embodiment of the invention of the first and second cutting blade having a different unexpanded configuration.

FIG. 13 shows a side view of the cutting blades of the bone cutting tool of an embodiment of the invention expanded into a cylindrical shape.

FIG. 14 shows a cross sectional view of an embodiment of the handle of the bone cutting tool of an embodiment of the invention tool is substantially perpendicular to a longitudinal axis.

FIGS. 15A-15B show flow charts of an embodiment of the method of the invention.

### DETAILED DESCRIPTION OF THE PREFERRED EMBODIMENTS

Devices for treating the spine, including those used in spinal fusion and spinal stabilization with motion preservation, are typically secured to the spine using screws which penetrate the bone. Such screws are designed to engage the structure of the bone. However, such bones may have been treated with bone cement in a prior procedure. For example, in a kyphoplasty or vertebroplasty procedure, bone cement is injected percutaneously into a fractured or degenerated vertebra with the goal of ameliorating vertebral compression fractures. The bone cement is injected into the bone where it fills natural or surgically created voids in the cancellous bone material within the bone.

A commonly used bone cement is polymethyl methacrylate or PMMA. Bone cements may include a powder (i.e., pre-polymerized PMMA and or PMMA or MMA co-polymer beads and/or amorphous powder, radio-opacifier, initiator) and a liquid (MMA monomer, stabilizer, inhibitor). Bone cements are typically provided as two-components which are mixed shortly before use. When the two components are mixed polymerization of the monomer begins. As polymerization continues the bone cement viscosity changes from a runny liquid into a dough-like state and then finally hardens into solid hardened material. The setting time can be tailored to provide suitable viscosity for implantation and help the physician safely apply the bone cement into the bone. A wide variety of bone cement formulations are known in the art.

Bone cement is implanted into bones in a variety of procedures using a variety of methods. For example, in kyphoplasty and vertebroplasty the bone cement is injected into the vertebra through a needle/cannula while liquid. In some procedures, the liquid bone cement is restrained to a particular portion of the bone using a barrier or barrier technique. In other procedures the liquid bone cement migrates through and fills natural voids in the cancellous bone. The net result is a bone that comprises portions of natural cancellous bone, and portions of cancellous bone embedded with bone cement.

Bone cement is a reliable anchorage and reinforcement material. It is easy to use in clinical practice and has a proven long survival rate with cemented-in prostheses. Moreover, the development of minimally invasive bone reinforcement procedures such as kyphoplasty and vertebroplasty has resulted in an increase of its use to reinforce the spine both as an adjunct to spinal stabilization procedures and as a therapy on its own. However, although bone cement is a hard stable material, it has properties different than the bone in which it resides. In particular, bone cement can be prone to fracture if disturbed after hardening/curing.

A situation that is arising with increasing frequency is the need to perform a spinal stabilization procedure (e.g. a spinal fusion or dynamic stabilization) on a spine in which the one or more vertebrae have been treated with bone cement. In such spinal stabilization procedures a spinal implant is anchored to two or more adjacent vertebrae. The spinal implant is designed to hold the adjacent vertebrae in fixed positions relative to one another to allow fusion or to stabilize and constrain the relative movement of the vertebrae and share the load between the vertebrae in dynamic stabilization. The implant is typically anchored to the vertebrae utilizing bone anchors, for example, bone screws which penetrate the bone. The bone screws are designed to engage and be secured to the natural bone structure including cortical and cancellous bone. However, bone screws are poorly adapted for use in bones which have been previously treated with bone cement. In particular, the use of bone screws in hardened bone cement can fracture the bone cement preventing the bone anchor from adequately securing the implant and degrading the reinforcing properties of the bone cement. Moreover, removing the hardened bone cement prior to the installing the anchor (and replacing with uncured bone cement) is time consuming and damaging to the integrity of the bone. Consequently, there is a need for new and improved devices and methods for securing spinal implants to vertebrae that have previously been treated with bone cement.

In embodiments of the present invention, a bone anchor, in the form of a bone screw, is provided which has different thread characteristics on the distal shaft adjacent the tip as compared to the proximal shaft adjacent the head. The thread on the proximal shaft is designed to engage and secure the anchor to natural cancellous and cortical bone. The thread on the distal shaft is designed to engage and secure the anchor to bone cement embedded within the bone.

In particular embodiments the bone anchor has more threads on the distal shaft than on the proximal shaft. The threads on the distal shaft merge into the thread(s) of the proximal shaft at the transition between the proximal and distal shafts. The increased number of threads on the distal shaft allows the depth of the thread to be reduced to a suitable depth for engaging bone cement without fracture while maintaining sufficient surface area for the distal threads to engage and secure the anchor to the bone cement.

The pitch of the threads on the distal shaft (distance between adjacent threads) and pitch of the thread(s) on the proximal shaft are selected to be consistent with the lead of the screw (the distance the screw advances along its axis during one complete turn). Thus, in one embodiment, the bone anchor has two distal threads on the distal shaft and one proximal thread on the proximal shaft. The thread pitch on the proximal shaft is equal to the lead. The thread pitch on the distal shaft is half of the thread pitch on the proximal shaft and, thus, equal to half of the lead. The reduced thread depth and thread pitch on the distal shaft results in thread characteristics similar to that of a machine screw on the distal shaft while maintaining thread characteristics on the proximal shaft more typical of a bone screw.

During implantation, a pilot bore is made into the vertebra passing through the natural cancellous and cortical bone and into the bone cement at the position at which the bone anchor is to be implanted. The pilot bore is made, for example, by a bone drill. The size of the pilot bore includes a distal bore sized to receive the distal shaft and a proximal bore sized to receive the proximal shaft (equal or typically larger in diameter than the distal shaft). The bone anchor is then inserted into the pilot bore such that the multiple distal threads engage the distal bore drawing the bone anchor into the pilot bore. Turning the bone anchor through one complete turn advances the bone anchor into the bore by a distance equivalent to the lead. As the bone anchor advances, the bone cement of the distal bore is engaged by the two threads on the distal shaft which have characteristics suitable for securing the distal shaft to the bone cement without fracturing it. The natural cancellous and cortical bone of the proximal bore is engaged by the single thread of the proximal shaft which has characteristics suitable for securing the proximal shaft to the bone.

Thus, embodiments of the invention provide a bone anchor shaft design suitable for anchoring an implant into a bone including bone cement. The shaft design can be applied to any type of bone anchor useful for bone surgery where it is to be used in a bone comprising bone cement including, but not limited to, lag screw, bone screws, pedicle screws, adjustable pedicle screws, polyaxial pedicle screws, dynamic bone screws, and Steffee screws.

These and other objects, features and advantages of the invention will be further apparent from the drawings and description of particular embodiments below. Common reference numerals are used to indicate like elements throughout the drawings and detailed description; therefore, reference numerals used in a drawing may or may not be referenced in the detailed description specific to such drawing if the associated element is described elsewhere. The first digit in a three digit reference numeral indicates the series of figures in which the referenced item first appears. Likewise, the first two digits in a four digit reference numeral.

The terms “vertical” and “horizontal” are used throughout the detailed description to describe general orientation of structures relative to the spine of a human patient that is standing. This application also uses the terms proximal and distal in the conventional manner when describing the components of the spinal implant system. Thus, proximal refers to the end or side of a device or component closest to the hand operating the device, whereas distal refers to the end or side of a device furthest from the hand operating the device. For example, the tip of a bone screw that enters a bone would conventionally be called the distal end (it is furthest from the surgeon) while the head of the screw would be termed the proximal end (it is closest to the surgeon).

### Bone Anchor

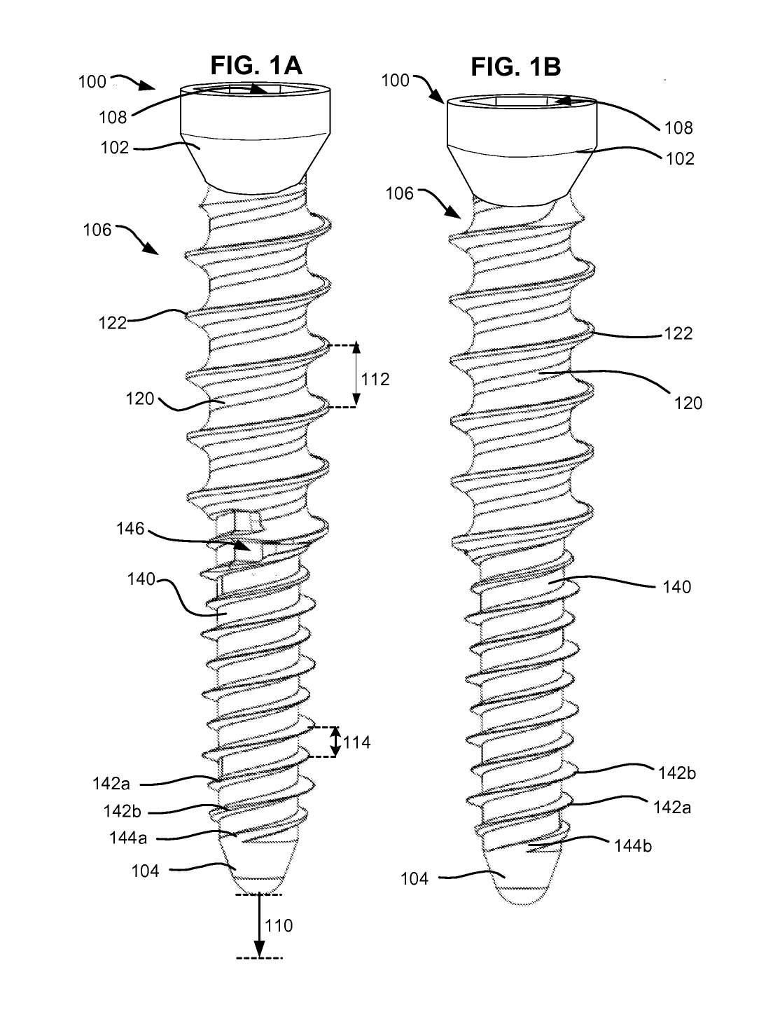

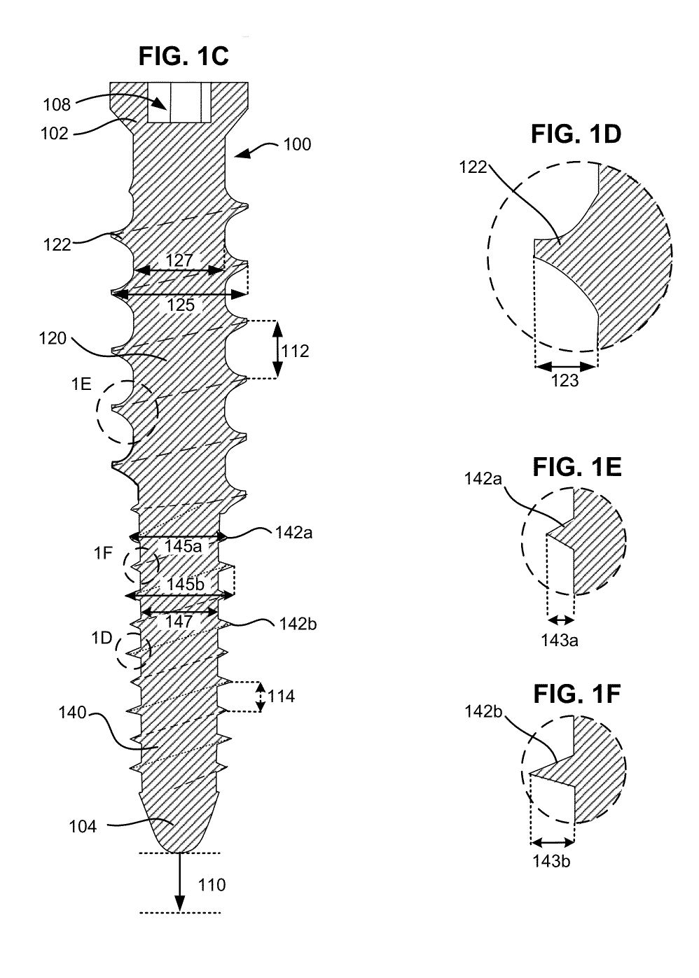

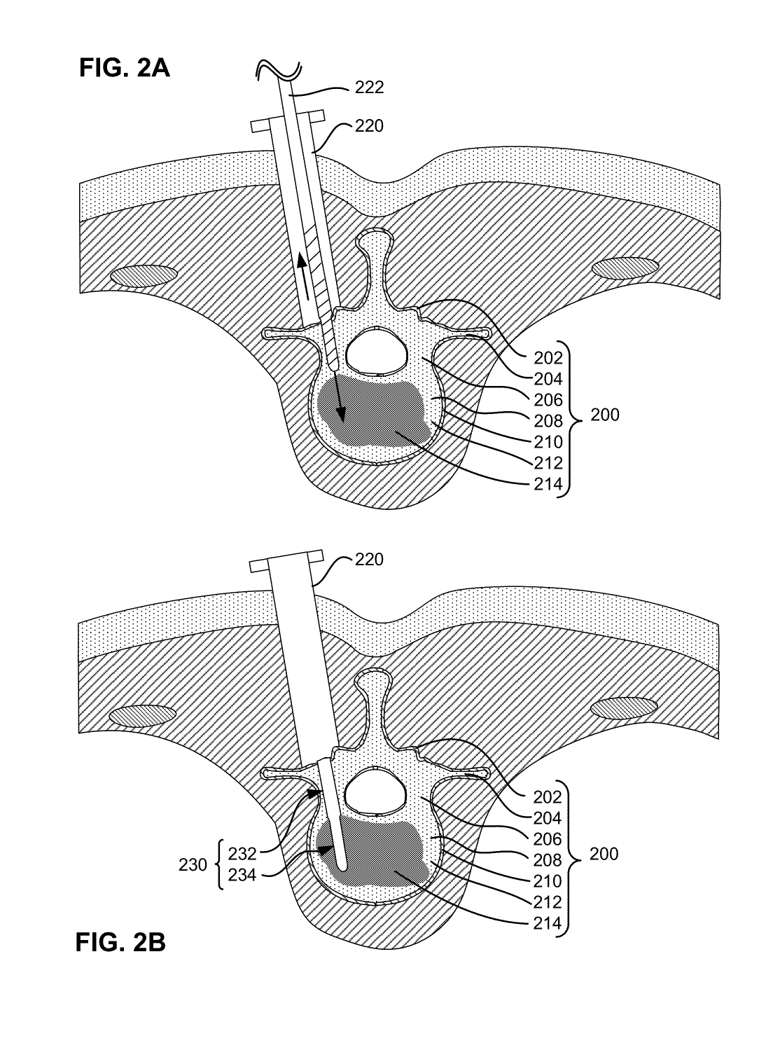

FIGS. 1A-1F illustrate a bone anchor 100 in the form of a bone screw adapted to engage bone and bone cement present in the bone. FIGS. 1A and 1B are front and back perspective views of a bone anchor 100 according to an embodiment of the present invention. FIG. 1C is a sectional view of the bone anchor 100 of FIGS. 1A and 1B. FIGS. 1D, 1E, and 1F are enlargements of portions of FIG. 1C illustrating the thread profile.

Referring first to FIGS. 1A and 1B which show front and back perspective views of a bone anchor 100 according to an embodiment of the present invention. Bone anchor 100 includes a head 102, at the proximal end and a tip 104 at the distal end. A shaft 106 extends between head 102 and tip 104 and includes a proximal shaft 120 and a distal shaft 140. Proximal shaft 120 bears on its outside surface a single proximal thread 122. Distal shaft 140 bears on its outside surface first and second distal threads 142a and 142b. First and second distal threads 142a and 142b begin on opposite sides of distal shaft 140 adjacent tip 104. First distal thread 142a begins at first start 144a shown in FIG. 1A. Second distal thread 142b begins at second start 144b shown in FIG. 1B. First and second distal threads 142a and 142b merge together and connect to single proximal thread 122 at transition 146 between distal shaft 140 and proximal shaft 120. The proximal thread 122 has a thread depth and threadform suitable for engaging bone and the proximal thread pitch 112 on the proximal shaft 120 is equal to the lead 110. The distal threads 142a and 142b have a thread depth and threadform suitable for engaging bone cement, and the distal thread pitch 114 on the distal shaft 140 is half of the proximal thread pitch 112 on the proximal shaft, and, thus, equal to half of the lead 110. The reduced thread depth and thread pitch on the distal shaft 140 results in thread characteristics similar to that of a machine screw while maintaining thread characteristics on the proximal shaft 120 more typical of a bone screw.

Head 102 is illustrated as a simple countersunk head having an internal hex socket 108. Hex socket 108 is adapted to be engaged by a driver to turn bone anchor 100 during implantation. In alternative embodiments head 102 is replaced by any other bone anchor head including, but not limited to, Steffee heads, hex heads, hex socket heads, Torx heads, breakaway heads, fixed heads, polyaxial heads, pedicle screw heads, angled heads, dynamic bone anchor heads or other heads desired to be securely mounted to a bone containing hardened bone cement.

Note that in alternative embodiments, the number and pitch of the proximal and distal threads may be varied. For example, a bone anchor shaft can comprise two proximal threads having pitch P and two pairs of distal threads having pitch P/2 where each pair of distal threads merges into one of the proximal threads at the transition between the distal shaft and the proximal shaft. Alternatively, a bone anchor shaft can comprise one proximal threads having pitch P and three distal threads having pitch P/3 where the three distal threads merge into the proximal thread at the transition between the distal shaft and the proximal shaft. In general, the distal shaft is provided with a greater number of threads having a smaller pitch (and typically a smaller thread depth) than the proximal shaft where the pitch of the proximal threads and distal threads is calculated to be consistent with the lead of the bone anchor (the distance the bone anchor advances per rotation).

Referring now to FIGS. 1C, 1D, 1E and 1F which show sectional views of bone anchor 100. FIG. 1C shows a longitudinal section of the entire bone anchor 100. FIG. 1D shows an enlarged view of portion 1D of FIG. 1C and illustrates the threadform of proximal thread 122. FIG. 1E shows an enlarged view of portion 1E of FIG. 1C and illustrates the threadform of first distal thread 142a. FIG. 1F shows an enlarged view of portion 1F of FIG. 1C and illustrates the threadform of second distal thread 142b. As illustrated in FIG. 1C, the proximal thread pitch 112 on the proximal shaft 120—the distance between adjacent crests of proximal thread 122—is equal to the lead 110. The distal thread pitch 114 on the distal shaft 140—the distance between the crest of first distal thread 142a and an adjacent crest of second distal thread 142b—is equal to half of lead 110.

As shown in FIG. 1D, the proximal thread 122 has a thread depth and threadform suitable for engaging bone. Proximal thread 122 has a buttress threadform and has a proximal thread depth (distance from crest to root) 123 suitable for engaging bone.

As shown in FIG. 1E, the first distal thread 142a has a thread depth and threadform suitable for engaging bone cement. First distal thread 142a has a triangular or V-shaped threadform. First distal thread 142a has a first distal thread depth (distance from crest to root) 143a suitable for engaging bone cement. In embodiments, first distal thread depth 143a is less than proximal thread depth 123. First distal thread depth 143a can be, for example, 75%, 60%, 50% 40% or less of proximal thread depth 123.

As shown in FIG. 1F, the second distal thread 142b has a thread depth and threadform suitable for engaging bone cement. Second distal thread 142b has a triangular or V-shaped threadform. Second distal thread 142b has a second distal thread depth (distance from crest to root) 143b suitable for engaging bone cement. In embodiments, second distal thread depth 143b is less than proximal thread depth 123. Second distal thread depth 143b can be, for example, 75%, 60%, 50% 40% or less of proximal thread depth 123. In the embodiment illustrated in FIGS. 1A-1F, second distal thread depth 143b is approximately 70% of proximal thread depth 123 whereas the first distal thread depth 143a is approximately 40% of proximal thread depth 123. However, in alternative embodiments, second distal thread depth 143b is greater than, less than or the same as first distal thread depth 143a.

The distal threads 142a and 142b have a thread depth and threadform suitable for engaging bone cement, and the distal thread pitch 114 on the distal shaft 140 is half of the proximal thread pitch 112 on the proximal shaft, and, thus, equal to half of the lead 110. The reduced thread depth and thread pitch on the distal shaft 140 results in thread characteristics similar to that of a machine screw while maintaining thread characteristics on the proximal shaft 120 more typical of a bone screw.

Referring again to FIG. 1C, the proximal thread 122 has a proximal major diameter 125 equal to maximum diameter of the proximal thread 122 (crest to crest measured perpendicular to the longitudinal axis of the bone anchor) and a proximal minor diameter 127 (root to root measured perpendicular to the longitudinal axis of the bone anchor). The proximal minor diameter 127 can be conceived as the diameter of the proximal shaft 120. The proximal major diameter is generally equal to the proximal minor diameter plus twice the proximal thread depth 123.

Referring again to FIG. 1C, the first distal thread 142a has a first distal major diameter 145a equal to the maximum diameter of the first distal thread 142a (crest to crest measured perpendicular to the longitudinal axis of the bone anchor) and a distal minor diameter 147 (root to root measured perpendicular to the longitudinal axis of the bone anchor). The distal minor diameter 147 can be conceived as the diameter of the distal shaft 140. The first distal major diameter 145a is generally equal to the distal minor diameter 147 plus twice the first distal thread depth 143a.

Referring again to FIG. 1C, the second distal thread 142b has a second distal major diameter 145b equal to the maximum diameter of the second distal thread 142a (crest to crest measured perpendicular to the longitudinal axis of the bone anchor) and a distal minor diameter 147 (root to root measured perpendicular to the longitudinal axis of the bone anchor). The second distal major diameter 145b is generally equal to the distal minor diameter 147 plus twice the second distal thread depth 143b. Because the root of the first distal thread 142a connects with the root of the second distal thread 142b, the first distal thread 142a and second distal thread 142b have the same distal minor diameter 147 which can be conceived as the diameter of the distal shaft 140. In this embodiment having different first and second distal major diameters reduces and/or redirects stress placed on the bone cement during implantation thereby reducing the risk of fracturing the bone cement. High and low distal threads, as shown, can serve to redirect stress along the axis of the bone anchor rather than outwardly from the bore into the bone cement thereby minimizing cracking or splitting of the bone cement.

It should be noted that, in the embodiment shown in FIGS. 1A-1F, the distal minor diameter 147 is substantially constant along the length of distal shaft 140. Likewise, the proximal minor diameter 127 is substantially constant along the length of proximal shaft 120. In alternative embodiments, one or both of the proximal shaft 120 and distal shaft 140 are conical such that the proximal minor diameter 127 and/or distal minor diameter 147 increases going from the tip 104 towards the head 102. In the embodiment shown in FIGS. 1A-1F, the proximal minor diameter 127 is greater than the distal minor diameter 147, but less than the second distal major diameter 145b. In alternative embodiments, the major diameter of the distal threads may be selected to be less than the minor diameter of the proximal threads such that the distal threads do not engage the proximal bore of a pilot bore during implantation.

The lengths and diameters of bone anchors are selected as appropriate for the anatomy of the bones into which they are implanted. In the particular case of pedicle screws, the screws are typically manufactured with a variety of shaft lengths in the range from 30 mm to 60 mm long and shaft diameters in the range from 5 mm to 8.5 mm suitable for the size of the vertebra and pedicle into which they are implanted. The thread depth, threadform, lead and pitch is selected such that the threads defined thereby are suitable for engaging bone and/or bone cement as required. For example, in a range of pedicle screw embodiments of the bone anchor 100, the proximal shaft has a length between about 10 and about 50 mm and a proximal minor diameter (proximal shaft diameter) between about 5 and about 8.5 mm, the proximal thread has a proximal thread depth between about 1 mm and about 2.5 mm, the distal shaft has a length between about 10 and about 50 mm and a distal minor diameter (distal shaft diameter) between about 5 mm and about 8.5 mm, the first distal thread has a first distal thread depth between about 0.4 mm and about 1.5 mm, the second distal thread has a second distal thread depth between about 0.4 mm and about 1.5 mm, the lead is between about 2 mm and about 5 mm, the proximal pitch is the same as the lead and the distal pitch is half of the lead. In a particular pedicle screw embodiment of the bone anchor 100, the proximal shaft has a length of 20 mm and a proximal minor diameter (proximal shaft diameter) of 5.2 mm, the proximal thread has a proximal major diameter of 8 mm (proximal thread depth is 1.4 mm), the distal shaft has a length of 20 mm and a distal minor diameter (distal shaft diameter) of 4.4 mm, the first distal thread has a first distal major diameter of 5.6 mm (first distal thread depth is 0.6 mm), the second distal thread has a second distal major diameter of 6.4 mm (second distal thread depth is 1.0 mm), the lead is 3.2 mm, the proximal pitch is 3.2 mm and the distal pitch is 1.6 mm.

### Method for Implanting Bone Anchor

The implantation of a bone anchor/bone screw into a vertebra is preferably performed in a minimally invasive manner and, thus, tools are provided to facilitate installation and assembly through cannulae. These tools can also be used in open procedures. One suitable minimally invasive approach to the lumbar spine is the paraspinal intermuscular approach. This approach is described, for example, in “The Paraspinal Sacraspinalis-Splitting Approach to the Lumber Spine,” by Leon L. Wiltse et al., The Journal of Bone & Joint Surgery, Vol. 50-A, No. 5, July 1968, which is incorporated herein by reference. In general, the patient is positioned prone. Incisions are made posterior to the vertebrae to be stabilized. The dorsal fascia is opened and the paraspinal muscle is split to expose the facet joints and lateral processes of the vertebra. Either a cannula is inserted to provide for port access (minimally invasive) or a larger incision is made with tissue refraction to expose the vertebra (open procedure).

Once the access to the implantation location on the vertebra has been obtained, a bore is made in the vertebra to receive the bone anchor. Where the bone anchor is a pedicle screw, the bore is placed lateral to the facet joints and angled in towards the vertebral body. The diameter and profile of the bore is selected to be compatible with the shaft of the bone anchor to be implanted. For example, the distal bore is sized to receive and be engaged by the distal shaft of the bone anchor, and the proximal bore is sized to receive and be engaged by the proximal shaft of the bone anchor. The bore is, in some cases, formed using a single device having the desired size and profile. In alternative embodiments, the distal bore is formed with a first device and then the proximal bore is enlarged with a second device. The diameter and length of the proximal and distal bore is selected based on the anatomy of the patient and the bone screw selected. In preferred embodiments one or more twist drills are utilized in conjunction with suction in order to remove bone cement and bone material cut by the drill. After forming the proximal and distal bore, the drill is removed.

The bone anchor is inserted into the proximal bore. A driver connected to the head of the bone anchor is then used to turn the bone anchor such that the distal threads engage the distal bore and the proximal threads engage the proximal bore. For each complete turn of the bone anchor, the bone anchor advances by a distance along its axis equal to the lead. The distal threads engage the distal bore without fracturing the bone cement. The bone anchor is turned until the head of the bone anchor is at the desired position relative to the surface of the bone and the distal shaft is engaged and secured to the bone cement surrounding the distal shaft and the proximal shaft is engaged and secured to the bone surrounding the proximal shaft. After implantation of the bone anchor, the driver is disconnected from the head of the bone anchor. Other components of a spinal implant system, for example spinal rods, can then be mounted to the vertebra by securing them to the head of the bone anchor.

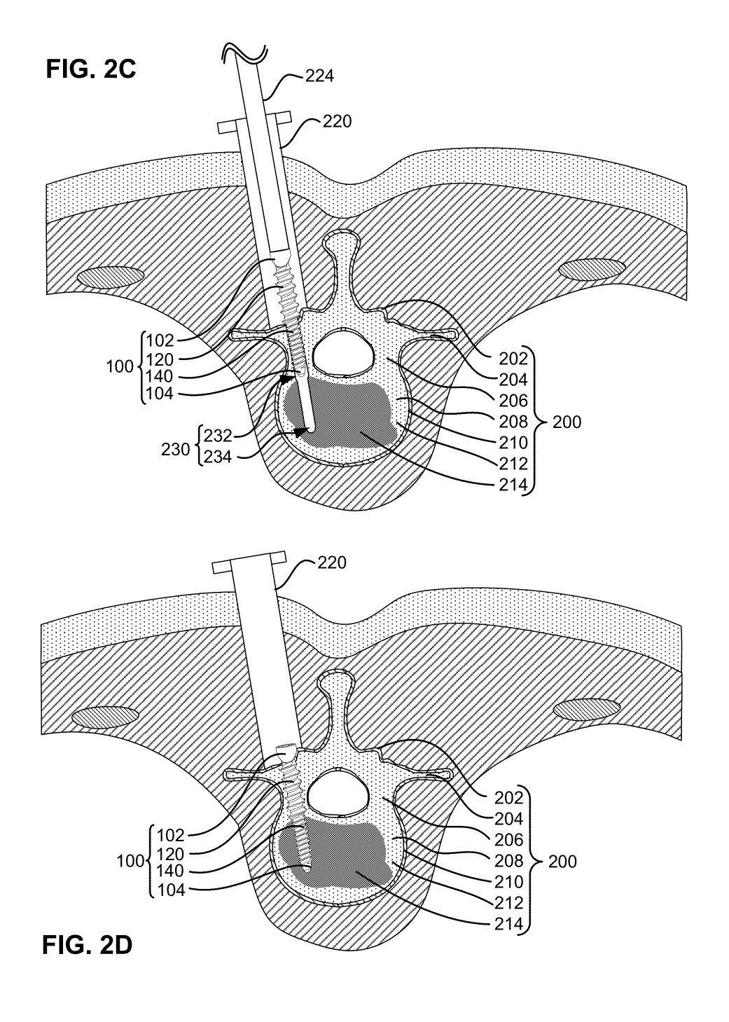

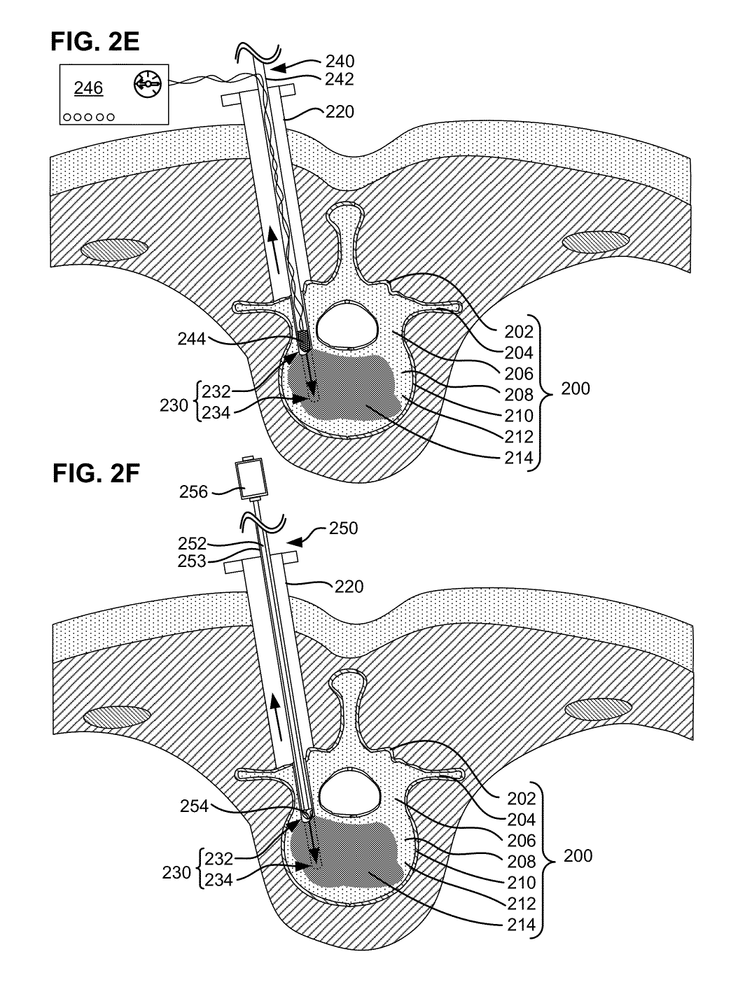

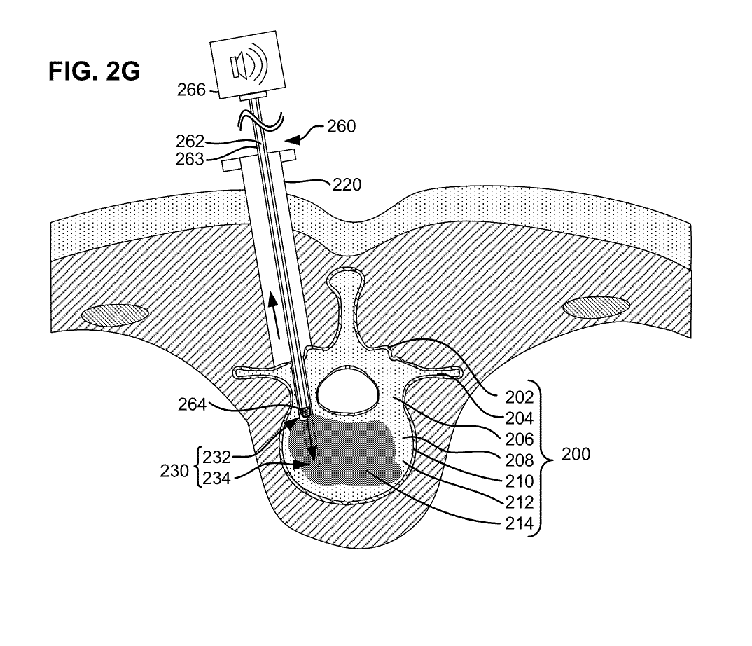

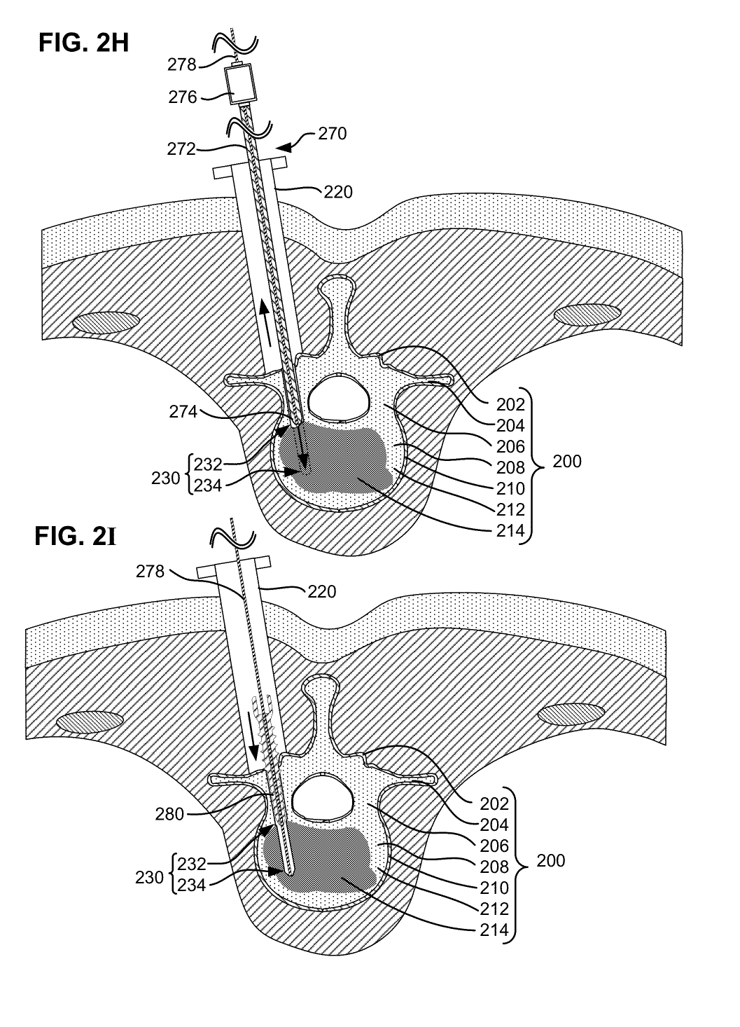

FIGS. 2A-2D show steps in the implantation of a bone anchor into a vertebra previously treated with bone cement. Referring first to FIG. 2A, the patient is positioned prone. Incisions are made posterior to the vertebrae 200. The dorsal fascia is opened and the paraspinal muscle is split to expose the facet joints 202 and lateral processes 204 of the vertebra 200. As shown, a cannula 220 is inserted to provide for port access. Alternatively, a larger incision is made with tissue retraction to expose the vertebra (open procedure). As shown, the vertebra 200 includes harder cortical bone 210 at the surface, spongy cancellous bone 212 in the interior, and hardened bone cement 214 within the vertebral body 208. Note that although bone cement 214 is shown for illustrative purposes as a homogenous mass, bone cement 214 may be distributed no-homogenously interspersed with regions including or consisting of cancellous bone.

Once the access to the implantation location on the vertebra 200 has been obtained, a bore is made in the vertebra 200 to receive to bone anchor. Where the bone anchor is a pedicle screw, the bore is placed lateral to the facet joints 202 and angled in towards the vertebral body 208. As shown in FIG. 2A, in one embodiment, a drill 222 having a stepped profile is inserted through the cannula 220 and advanced into the vertebra 200 through the cancellous bone 212 of the pedicle 206 and into the bone cement 214 within vertebral body 208. In alternative embodiments, two devices/drills are used in separate steps—the distal bore is formed with a first device and then the proximal bore is enlarged with a second device. Alternatively, the proximal bore is formed first with a first device (such as a blunt probe) through cancellous bone and the distal bore is created as an extension of the proximal bore into bone cement with an appropriate tool. In preferred embodiments, one or more low speed twist drills are utilized in conjunction with suction in order to remove bone cement and bone material cut by the drill. After forming the proximal and distal bore, the drill is removed.

In an alternative preferred embodiment a blunt probe is inserted through the pedicle to create the proximal bore. The probe can be passed through the pedicle without excessive force until it contacts bone cement. The probe compresses cancellous bone (enhancing bone density) rather than cutting and removing the bone. The length of probe in the pedicle, when it contacts the bone cement, can be assessed with fluoroscopy/radiographic imaging or markings on the probe or a gauge. The distal bore is then created using a twist drill which cuts away and removes bone cement from the distal bore. Suction is used to clean cut bone cement from the operative site prior to implantation of the screw. Radiographic imaging and/or a gauge is utilized to select the correct length of distal shaft. The length of the proximal bore and the length of the distal bore are assessed and used to select a bone anchor having a proximal shaft and distal shaft of the correct length for the patient's anatomy from a kit containing a variety of configurations of bone anchors.

The bone anchors are preferably provided in the form of a kit which includes a range of bone anchors having different lengths including different lengths of the proximal and distal shafts. Thus, a screw having a particular length of proximal shaft and distal shaft is selected as appropriate for the anatomy of the patient and the distribution of bone cement within the target vertebra. In some cases, imaging of the vertebra and bone cement within it may be used to preoperatively assess configurations of the bone anchor shaft (diameters and shaft length) suitable for implantation in order to ensure that a suitable variety of bone anchors are available for the procedure. In preferred embodiments, the kit and/or a separate toolkit includes a range of installation/implantation tools (as, for example, described herein) suitable for creation of the bore in a bone containing hardened bone cement and for implantation of the bone anchor in the bore thereby created.

As shown, in FIG. 2B, after forming the bore 230, the drill 222 is removed. The diameter and profile of the bore 230 is selected to be compatible with the patient's anatomy and the shaft of the bone anchor to be implanted. As shown in FIG. 2B, for example, the distal bore 234 within bone cement 214 is of a lower diameter sized to receive and be engaged by the distal shaft of the bone anchor, and the proximal bore 232 within cancellous bone 212 and cortical bone 210 is of a larger diameter sized to receive and be engaged by the proximal shaft of the bone anchor.

In embodiments, the relative lengths of the proximal and distal bore are selected based on the patient's anatomy and the position of the bone cement 214 within the vertebra 200. The position of the bone cement 214 within the vertebra 200 and the size of vertebra 200 are, in some cases, assessed using imaging during preoperative planning in order to select a bone anchor having appropriate characteristics, and, thus, determine the proper characteristics for the proximal bore 232 and distal bore 234. Alternatively, the size of the vertebra and position of the bone cement is assessed by the surgeon during the procedure using appropriate tools.

As shown, in FIG. 2C, the bone anchor 100 is inserted into the proximal bore 232. A driver 224 connected to the head 102 of the bone anchor 100 is then used to turn the bone anchor 100 such that the distal threads engage the distal bore 234 and the proximal threads engage the proximal bore 232. For each complete turn of the bone anchor 100, the bone anchor 100 advances by a distance along its axis equal to the lead. The distal threads engage the distal bore 234 without fracturing the bone cement 214. The bone anchor 100 is turned until the head 102 of the bone anchor 100 is at the desired position relative to the surface of the vertebra 200 and the distal shaft 140 is engaged and secured to the bone cement 214 surrounding the distal bore 234 and the proximal shaft 120 is engaged and secured to the cancellous bone 212 and cortical bone 210 surrounding the proximal bore 232.

As shown in FIG. 2D, after implantation of the bone anchor 100 into the vertebra 200 the driver is disconnected from the head 102 of the bone anchor 100. Other components of a spinal implant system, for example, spinal rods, can then be mounted to the vertebra by securing them to the head 102 of the bone anchor 100.

### Alternative Implantation Procedures

As illustrated above in FIG. 2A, and described in the accompanying text a bore 230 (including a proximal bore 232 and a distal bore 234) is created in a vertebra to receive a bone anchor. One way of creating the bore 230 is with one or more drills or with a blunt probe and a drill. However, the distal bore 234 in the bone cement can be created using a variety of techniques and devices. The most common bone cement, PMMA, is a hard glass-like polymer which can be prone to fracture when drilled or machined. However, because of the particular properties of bone cement/PMMA, a bore can be made in PMMA using a number of techniques unsuitable for creating a bore in bone. Thus, in some embodiments, the distal bore 234 is created using a different method and apparatus than used to create the proximal bore 232. For example, the glass transition temperature of PMMA ranges from 85° C. to 165° C. or more depending upon the formulation. PMMA may safely be heated above its glass transition temperature before, during and/or after manipulation to soften and/or melt the PMMA in order to reduce the risk of fracture.

In one method, a heated probe is used to melt the PMMA. The melted PMMA can be displaced or removed during insertion of the heated probe. The probe can be heated electrically, ultrasonically, mechanically or using electromagnetic radiation such as, for example, a laser. Alternatively, the distal bore is created using a mechanical tool such as a rotating burr that mechanically heats the PMMA and softens/melts the PMMA during creation of the bore. Alternatively, the distal bore is created using a drill and then the bone cement surrounding the distal bore is heat treated before or during bone anchor implantation to anneal/fuse any fractures that may have been formed during the cutting of the distal bore. Alternatively, an ultrasound probe can be used to heat and soften the bone cement during creation of the distal bore.

FIG. 2E illustrates an alternative method for creating distal bore 234. As before, the proximal bore 232 is created using conventional methods for creating a bore in a vertebra, e.g. a blunt probe or drill. For example, a probe can be passed through the pedicle without excessive force until it contacts bone cement. When the probe contacts bone cement, it is removed and a heated probe 240 is inserted through cannula 220. Heated probe 240 includes a shaft 242 and heated tip 244. A power/temperature controller 246 is coupled to heated tip 244 through shaft 242. The power/temperature controller 246 provides one of electrical, ultrasonic or electromagnetic energy to heat heated tip 244. In some embodiments, heated probe 240 is inserted through a hollow sleeve (not shown). The hollow sleeve is inserted into and engages the proximal bore 232, aligns the heated tip 244 with the distal bore 234, and insulates the bone adjacent proximal bore 232 from heating by heated tip 244.

In use, the physician operates power/temperature controller 246 to raise the temperature of heated tip 244 above the glass transition temperature of bone cement 214. The physician utilizes shaft 242 to drive heated tip 244 into bone cement 214. Bone cement 214 flows away from heated tip 244 as heated tip 244 is introduced creating distal bore 234 (dotted lines). Heated probe 240 is, in some embodiments, provided with channels and/or grooves which allow melted bone cement 214 to flow towards the proximal bore 232. When a distal bore 234 having a desired length as been created, heated probe 240 is removed. Heated tip 244 and bone cement 214 may be allowed to cool prior to removal of heated probe 240 in order that melted bone cement 214 does not flow into distal bore 234 after removal of heated probe 240.

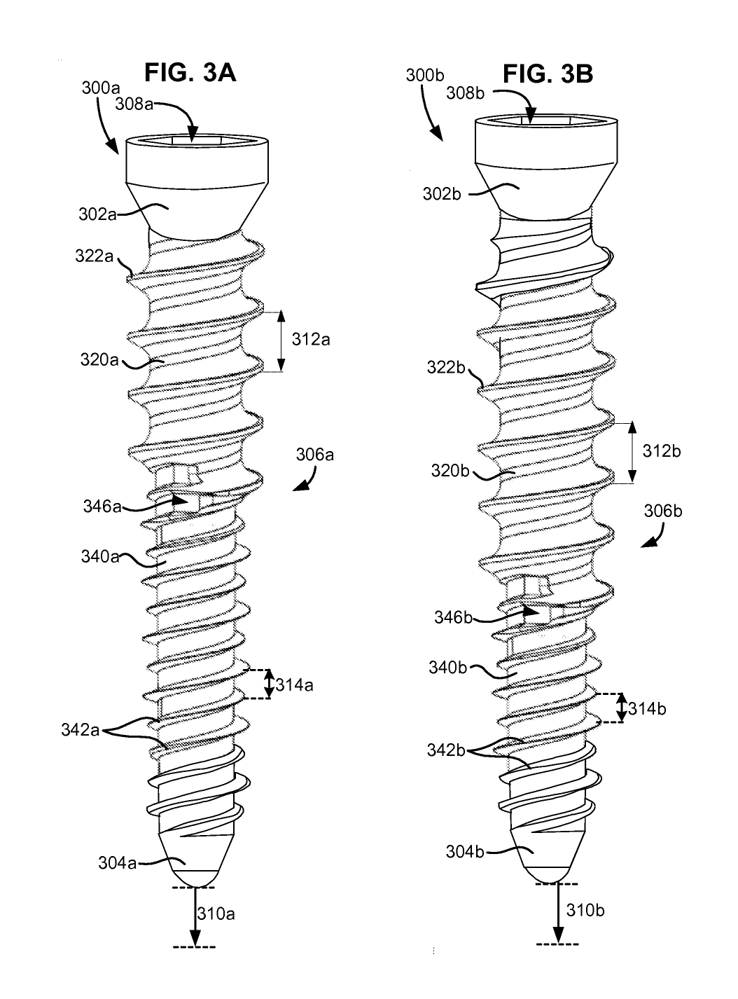

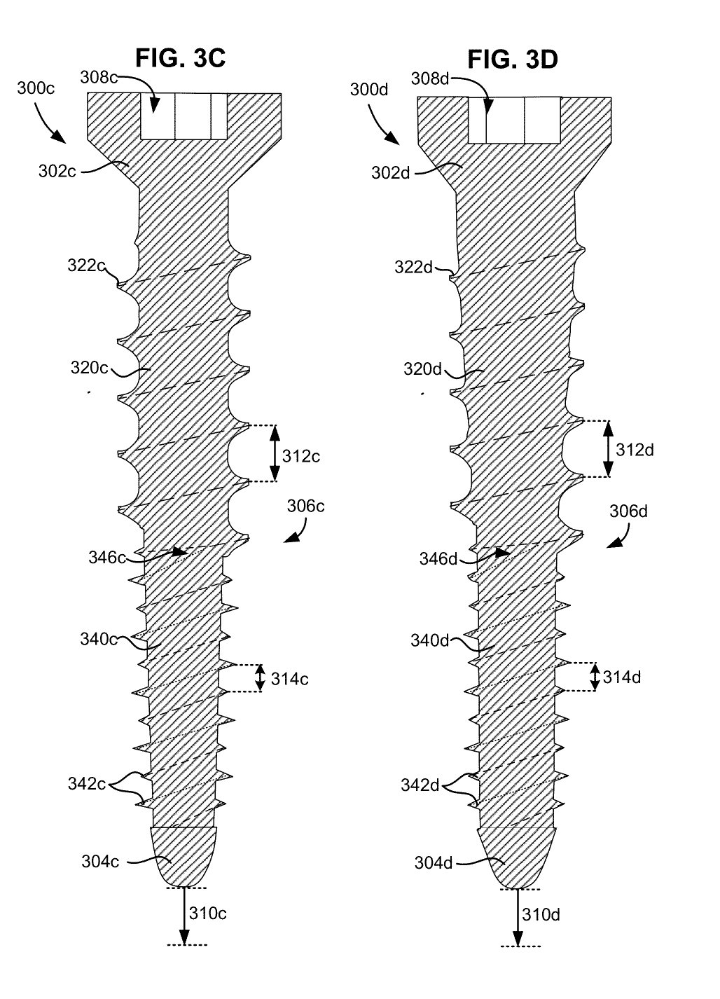

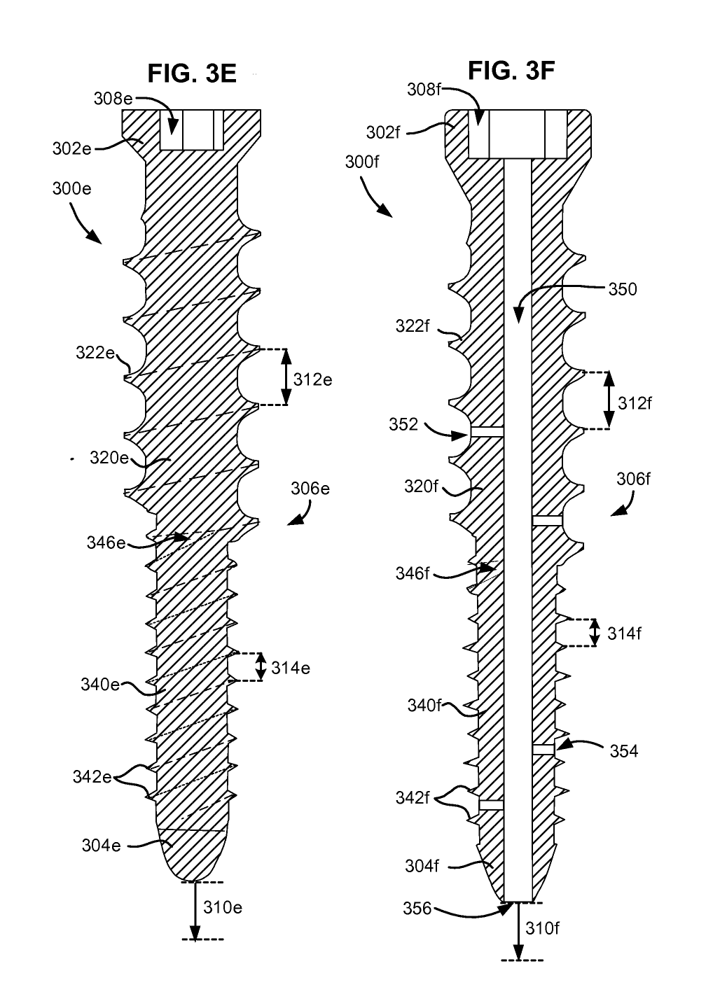

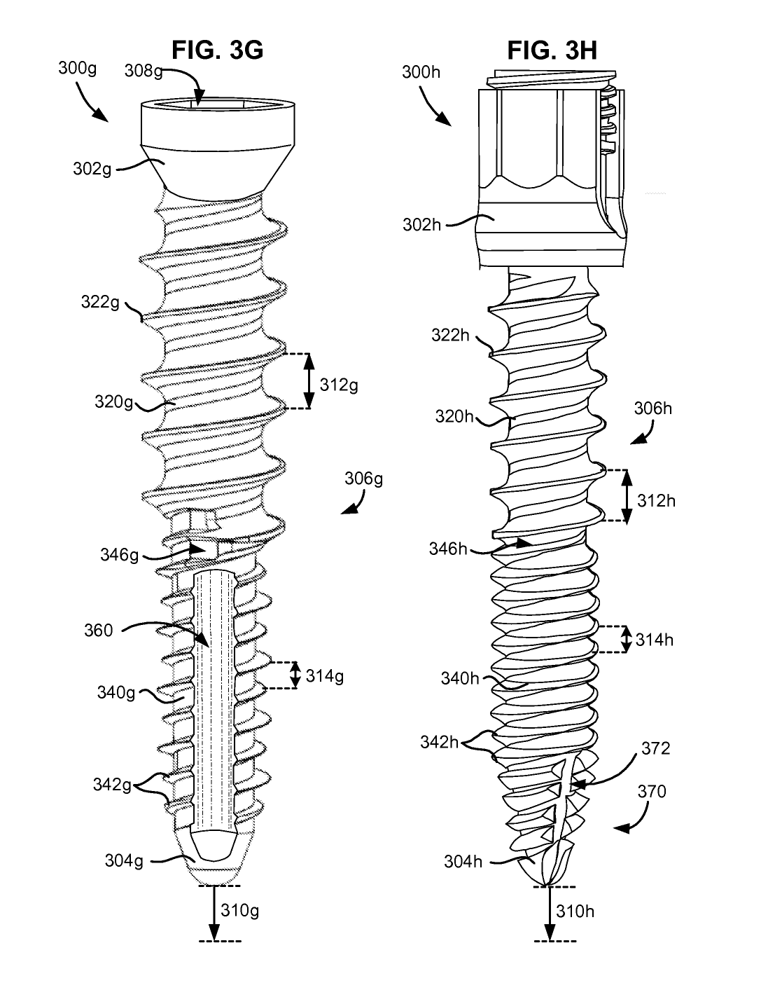

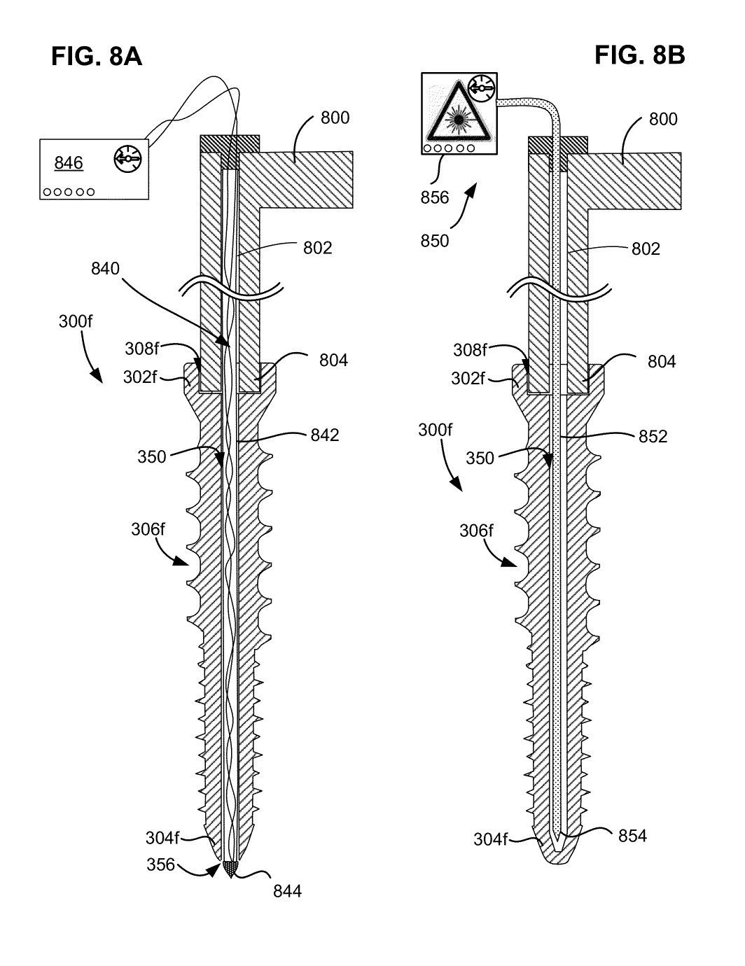

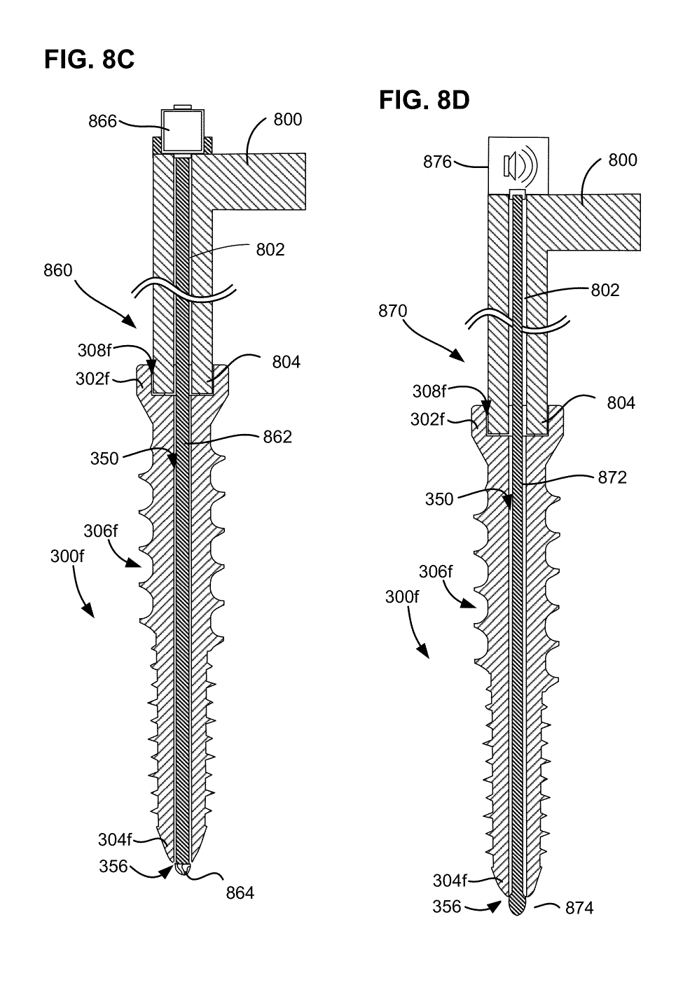

In an alternative embodiment heated probe 240 is inserted through a cannulated bone anchor (see e.g. FIG. 3F) such that heated tip 244 extends beyond the distal end of the bone anchor (See, e.g. FIGS. 8A-8C). In this procedure heated tip 244 is used to melt the bone cement during implantation of the bone anchor thereby reducing the possibility of fracture. In this embodiment, distal bore 234 may be formed simultaneously with the implantation of the bone anchor.

FIG. 2F illustrates an alternative method for creating distal bore 234. As before, the proximal bore 232 is created using conventional methods for creating a bore in a vertebra, e.g. a blunt probe or drill. For example, a probe can be passed through the pedicle without excessive force until it contacts bone cement. When the probe contacts bone cement, it is removed and a rotary probe 250 is inserted through cannula 220. Rotary probe 250 includes a shaft 252 and burr tip 254. A driver 256 (for example, an electrical motor) is coupled to burr tip 254 through shaft 252. The driver 256 rotates shaft 252 and burr tip 254 at high speed. In some embodiments, rotary probe 250 includes a hollow sleeve 253 through which shaft 252 passes. The hollow sleeve 253 is inserted into and engages the proximal bore 232, aligns the burr tip 254 with the distal bore 234, and prevents contact between shaft 252 and the bone adjacent proximal bore 232.

In use, the physician operates driver 256 to rotate the burr tip 254 at high speed. Friction between burr tip 254 and bone cement 214 raises the temperature of burr tip 254 and bone cement 214 above the glass transition temperature of bone cement 214. The physician utilizes shaft 252 to drive burr tip 254 into bone cement 214. Bone cement 214 flows away from burr tip 254 as burr tip 254 is introduced creating distal bore 234 (dotted lines). Rotary probe 250 is, in some embodiments, provided with channels and/or grooves which allow melted bone cement 214 to flow towards the proximal bore 232. When a distal bore 234 having a desired length as been created, rotary probe 250 is removed. Burr tip 254 and bone cement 214 may be allowed to cool prior to removal of rotary probe 250 in order that melted bone cement 214 does not flow into distal bore 234 after removal of rotary probe 250.

In an alternative embodiment rotary probe 250 is inserted through a cannulated bone anchor (see e.g. FIG. 3F) such that burr tip 254 extends beyond the distal end of the bone anchor (See, e.g. FIGS. 8A-8C). In this procedure burr tip 254 is used to melt the bone cement during implantation of the bone anchor thereby reducing the possibility of fracture. In this embodiment, distal bore 234 may be formed simultaneously with the implantation of the bone anchor.

FIG. 2G illustrates an alternative method for creating distal bore 234. As before, the proximal bore 232 is created using conventional methods for creating a bore in a vertebra, e.g. a blunt probe or drill. For example, a probe can be passed through the pedicle without excessive force until it contacts bone cement. When the probe contacts bone cement, it is removed and an ultrasonic probe 260 is inserted through cannula 220. Ultrasonic probe 260 includes a shaft 262 and ultrasonic tip 264. An ultrasonic transducer 266 is coupled to ultrasonic tip 264 through shaft 262. The ultrasonic transducer 266 provides ultrasonic vibrations through shaft 262 to ultrasonic tip 264. In some embodiments, ultrasonic probe 260 includes a hollow sleeve 263 through which shaft 262 passes. The hollow sleeve 263 is inserted into and engages the proximal bore 232, aligns the ultrasonic tip 264 with the distal bore 234, and prevents contact between shaft 262 and the bone adjacent proximal bore 232.

In use, the physician operates ultrasonic transducer 266 to vibrate the ultrasonic tip 264 at high frequency. High frequency vibration where the ultrasonic tip 264 contacts bone cement 214 raises the temperature of ultrasonic tip 264 and bone cement 214 above the glass transition temperature of bone cement 214. The physician utilizes shaft 262 to drive ultrasonic tip 264 into bone cement 214. Bone cement 214 flows away from ultrasonic tip 264 as ultrasonic tip 264 is introduced—creating distal bore 234 (dotted lines). Ultrasonic probe 260 is, in some embodiments, provided with channels and/or grooves which allow melted bone cement 214 to flow towards the proximal bore 232. When a distal bore 234 having a desired length has been created, ultrasonic probe 260 is removed. Ultrasonic tip 264 and bone cement 214 may be allowed to cool prior to removal of ultrasonic probe 260 in order that melted bone cement 214 does not flow into distal bore 234 after removal of ultrasonic probe 260.

In an alternative embodiment ultrasonic probe 260 is inserted through a cannulated bone anchor (see e.g. FIG. 3F) such that ultrasonic tip 264 extends beyond the distal end of the bone anchor (see, e.g. FIGS. 8A-8D). In this procedure, ultrasonic tip 264 is used to melt the bone cement during implantation of the bone anchor thereby reducing the possibility of fracture. In this embodiment, distal bore 234 may be formed simultaneously with the implantation of the bone anchor.

The tools for creating the proximal and/or distal bore are, in some embodiments, cannulated such that they are adapted to be received over a guide wire to facilitate proper location of the tools relative to the bone during bore formation. In such a procedure a wire, for example a k-wire or other guidewire, is positioned at the target position on or in the bone, the cannulated bore creation tool is then directed over the guidewire to the target position. The guidewire is received in the central bore of the cannulated bore creation tool. The cannulated bore creation tool is then used to create and/or extend the bore. The guidewire is advanced with or incrementally ahead of the bore creation tool as the bore is created and/or extended. When a bore of the desired size has been created, the cannulated bore creation tool is withdrawn leaving the guidewire in place. If necessary or desirable, additional tools may be inserted over the guidewire to prepare the bore for implantation of a bone anchor and removed subsequent to use while maintaining the guidewire within the bore. When the desired bore has been prepared, a cannulated bone anchor is inserted over the guidewire and thereby directed to the bore for implantation. The guidewire is removed after the bone anchor is implanted at the correct position.

Maintaining the guidewire at the target location and within the bore facilitates the implantation procedure by ensuring a consistent location and orientation of the tool(s) and bone anchor during the procedure. This is particularly useful where the procedure is minimally invasive and/or percutaneous where the physician may not have direct visualization of the bone. Radiographic/fluoroscopic imaging can be used during initial placement of the guidewire. Thereafter, the placement of the guidewire is maintained and used to orient the tools and bone anchor, and, thus, the need for additional radiographic/fluoroscopic imaging during subsequent steps is reduced and/or eliminated thereby reducing procedure time and/or physician exposure to radiation.

Each of the tools for bore creation described herein can be cannulated in order to allow for use of a guidewire including, but not limited to, a heated probe, ultrasound probe, blunt probe, drill, stepped drill, burr probe, thermoelectric probe, or laser heated probe. FIGS. 2H and 2I illustrate two of the steps in the use of guidewire to guide implantation of a bone anchor. As shown in FIG. 2H, a guidewire 278 is positioned relative to vertebra 200 and aligned with the longitudinal axis of bore 230. A cannulated bore creation tool 270 having a cannulated shaft 272 is received over guidewire 278. The guidewire 278 is received in a central bore of the cannulated bore creation tool 270 which can slide along the guidewire 278. The driver 276 (for example, a motor) is used to operate head 274 (for example, a burr tip or drill) via the cannulated shaft 272 to create (in this step) distal bore 234 by extending proximal bore 232. The guidewire 278 is advanced with the cannulated bore creation tool 270 as the bore is extended. (The proximal bore can be created the same way.) If necessary or desirable, the cannulated bore creation tool 270 may be exchanged with a different cannulated bore creation tool 270 to prepare the bore 230 while maintaining the guidewire 278 in place within the bore 230. For example, a cannulated thread tapping tool (not shown) may be used to create threads in the bore 230—the tap may be inserted over the guidewire, used to create threads in the bore 230, and then removed, leaving the guidewire 278 in place within the bore 230.

When the desired bore 230 has been prepared, the cannulated bore creation tool(s) 270 is/are removed leaving the guidewire 278 in position and aligned with the bore 230 as shown in FIG. 2I. A cannulated bone anchor 280 (see e.g. FIG. 3F) is then placed on guidewire 278. The physician slides cannulated bone anchor 280 along guidewire 278 which directs the cannulated bone anchor 280 to bore 230 and aligns cannulated bone anchor 280 with bore 230. The cannulated bone anchor 280 is then implanted in the bore 230 using a driver appropriate to the cannulated bone anchor 280 (the driver is, in some embodiments, also received over guidewire 278). The guidewire is removed after the cannulated bone anchor 280 is implanted at the correct position with bore 230 and vertebra 200. In some embodiments, the guidewire may also be used to guide installation of additional spinal components by guiding connection of the components to the head of cannulated bone anchor 280.

### Variations of Bone Anchor Shaft

FIGS. 3A-3H illustrate variations of the shaft of the bone anchor 100 of FIGS. 1A-1F. As previously described, the shaft of the bone anchor including proximal shaft 120 and distal shaft 140 is designed/selected to be compatible with the anatomy of the bone into which it is to be implanted and the relative positions and extent of bone cement and natural bone material within the bone. In preferred embodiments, both the proximal and distal shafts are cylindrical with the proximal shaft having a larger diameter than the distal shaft. In alternative embodiments one or more of the proximal shaft and distal shaft is tapered/conical. The thread depth can also be varied over the length of one or more of the proximal shaft and distal shaft. Moreover, the relative lengths of the proximal shaft and distal shaft and the overall length of the bone anchor are varied so as to be suitable for bones of different sizes and having different positions and extent of bone cement and natural bone material within the bone. The bone anchors may be provided in the form of a kit which includes a range of bone anchors having different features and different lengths including different lengths of the proximal and distal shafts. The physician is, thus, able to select from the kit, during the procedure, bone anchors suitable for the particular anatomy of the bone in which a bone anchor is desired to be implanted.

FIG. 3A shows a perspective view of a bone anchor 300a according to an alternative embodiment of the present invention. Bone anchor 300a includes a head 302a, at the proximal end and a tip 304a at the distal end. A shaft 306a extends between head 302a and tip 304a and includes a proximal shaft 320a and a distal shaft 340a. Proximal shaft 320a bears on its outside surface a single proximal thread 322a. Distal shaft 340a bears on its outside surface first and second distal threads 342a. First and second distal threads 342a merge together and connect to single proximal thread 322a at the transition 346a between the distal shaft 340a and proximal shaft 320a. The proximal thread 322a has a thread depth and threadform suitable for engaging bone and the proximal thread pitch 312a on the proximal shaft 320a is equal to the lead 310a. The distal threads 342a have a thread depth and threadform suitable for engaging bone cement and the distal thread pitch 314a on the distal shaft 340a is half of the proximal thread pitch 312a on the proximal shaft 320a, and, thus, equal to half of the lead 310a. In the alternative embodiment shown in FIG. 3A, the length of proximal shaft 320a is reduced and the length of distal shaft 340a is increased relative to the embodiment of FIGS. 1A-1F. The alternative bone anchor 300a of FIG. 3A is, thus, suited to implantation in a bone having a larger extent of bone cement in which distal shaft 340a is to be secured.

FIG. 3B shows a perspective view of a bone anchor 300b according to an alternative embodiment of the present invention. Bone anchor 300b includes a head 302b, at the proximal end and a tip 304b at the distal end. A shaft 306b extends between head 302b and tip 304b and includes a proximal shaft 320b and a distal shaft 340b. Proximal shaft 320b bears on its outside surface a single proximal thread 322b. Distal shaft 340b bears on its outside surface first and second distal threads 342b. First and second distal threads 342b merge together and connect to single proximal thread 322b at the transition 346b between the distal shaft 340b and proximal shaft 320b. The proximal thread 322b has a thread depth and threadform suitable for engaging bone and the proximal thread pitch 312b on the proximal shaft 320b is equal to the lead 310b. The distal threads 342b have a thread depth and threadform suitable for engaging bone cement and the distal thread pitch 314b on the distal shaft 340b is half of the proximal thread pitch 312b on the proximal shaft 320b, and, thus, equal to half of the lead 310b. In the alternative embodiment shown in FIG. 3B, the length of proximal shaft 320b is increased and the length of distal shaft 340b is reduced relative to the embodiment of FIGS. 1A-1F. The alternative bone anchor 300b of FIG. 3A is, thus, suited to implantation in a bone having a smaller extent of bone cement in which distal shaft 340b is to be secured.

FIG. 3C shows a sectional view of a bone anchor 300c according to an alternative embodiment of the present invention. Bone anchor 300c includes a head 302c, at the proximal end and a tip 304c at the distal end. A shaft 306c extends between head 302c and tip 304c and includes a proximal shaft 320c and a distal shaft 340c. Proximal shaft 320c bears on its outside surface a single proximal thread 322c. Distal shaft 340c bears on its outside surface first and second distal threads 342c. First and second distal threads 342c merge together and connect to single proximal thread 322c at the transition 346c between the distal shaft 340c and proximal shaft 320c. The proximal thread 322c has a thread depth and threadform suitable for engaging bone and the proximal thread pitch 312c on the proximal shaft 320c is equal to the lead 310c. The distal threads 342c have a thread depth and threadform suitable for engaging bone cement and the distal thread pitch 314c on the distal shaft 340c is half of the proximal thread pitch 312c on the proximal shaft 320c and, thus, equal to half of the lead 310c. In the alternative embodiment shown in FIG. 3C, distal shaft 340c is tapered/conical in that the minor diameter of the distal shaft increases going from tip 304c towards transition 346c. In the embodiment shown, the thread depth of the distal threads remains constant over the distal shaft 340c, thus, the major diameter of the distal shaft also increases going from tip 304c towards transition 346c. Proximal shaft 320c is, in this embodiment, cylindrical, and has a minor diameter greater than or equal to the maximum minor diameter of the distal shaft 340c. In alternative embodiments, proximal shaft 320c is also conical in shape.

FIG. 3D shows views of a bone anchor 300d according to an alternative embodiment of the present invention. Bone anchor 300d includes a head 302d, at the proximal end and a tip 304d at the distal end. A shaft 306d extends between head 302d and tip 304d and includes a proximal shaft 320d and a distal shaft 340d. Proximal shaft 320d bears on its outside surface a single proximal thread 322d. Distal shaft 340d bears on its outside surface first and second distal threads 342d. First and second distal threads 342d merge together and connect to single proximal thread 322d at the transition 346d between the distal shaft 340d and proximal shaft 320d. The proximal thread 322d has a thread depth and threadform suitable for engaging bone and the proximal thread pitch 312d on the proximal shaft 320d is equal to the lead 310d. The distal threads 342d have a thread depth and threadform suitable for engaging bone cement, and the distal thread pitch 314d on the distal shaft 340d is half of the proximal thread pitch 312d on the proximal shaft 320d, and, thus, equal to half of the lead 310d. In the alternative embodiment shown in FIG. 3D, the proximal shaft 320d is conical/tapered and increases in minor diameter going from transition 346d towards head 302d. Note however, that the major diameter of proximal shaft 320d is substantially constant such that as the shaft increases in diameter going towards head 302d, the thread depth of the proximal thread is reduced. The conical design of proximal shaft 320d serves to compress cancellous and cortical bone surrounding the proximal bore which can assist the engagement between proximal thread 322d and the bone.

FIG. 3E shows a section view of a bone anchor 300e according to an alternative embodiment of the present invention. Bone anchor 300e includes a head 302e, at the proximal end and a tip 304e at the distal end. A shaft 306e extends between head 302e and tip 304e and includes a proximal shaft 320e and a distal shaft 340e. Proximal shaft 320e bears on its outside surface a single proximal thread 322e. Distal shaft 340e bears on its outside surface first and second distal threads 342e. First and second distal threads 342e merge together and connect to single proximal thread 322e at the transition 346e between the distal shaft 340e and proximal shaft 320e. The proximal thread 322e has a thread depth and threadform suitable for engaging bone and the proximal thread pitch 312e on the proximal shaft 320e is equal to the lead 310e. The distal threads 342e have a thread depth and threadform suitable for engaging bone cement and the distal thread pitch 314e on the distal shaft 340e is half of the proximal thread pitch 312e on the proximal shaft 320e, and, thus, equal to half of the lead 310e. In the alternative embodiment shown in FIG. 3E, the thread depth and threadform of both of distal threads 342e is identical. Moreover, the major diameter of distal shaft (crest to crest) is less than the minor diameter of the proximal shaft. Thus, distal shaft 340e can pass through a proximal bore of suitable diameter for proximal shaft 320e without distal threads 342e engaging the wall of the proximal bore thereby facilitating implantation of bone anchor 300e.

FIG. 3F which shows views of a bone anchor 300f according to an alternative embodiment of the present invention. Bone anchor 300f includes a head 302f, at the proximal end and a tip 304f at the distal end. A shaft 306f extends between head 302f and tip 304f and includes a proximal shaft 320f and a distal shaft 340f. Proximal shaft 320f bears on its outside surface a single proximal thread 322f. Distal shaft 340f bears on its outside surface first and second distal threads 342f. First and second distal threads 342f merge together and connect to single proximal thread 322f at the transition 346f between the distal shaft 340f and proximal shaft 320f. The proximal thread 322f has a thread depth and threadform suitable for engaging bone and the proximal thread pitch 312f on the proximal shaft 320f is equal to the lead 310f. The distal threads 342f have a thread depth and threadform suitable for engaging bone cement and the distal thread pitch 314f on the distal shaft 340f is half of the proximal thread pitch 312f on the proximal shaft 320f, and, thus, equal to half of the lead 310f.

In the alternative embodiment shown in FIG. 3F, bone anchor 300f is cannulated in that a continuous bore 350 extends through head 302f, proximal shaft 320f, distal shaft 340f and tip 304f. The continuous bore 350 can be sized to receive a guidewire such that bone anchor 300f can be guided to a target location over a guidewire. Also, continuous bore 350 can be utilized for the injection of bone cement into the bone to strengthen bone and/or connection between the bone and the bone anchor 300f after implantation. Bone cement injected through head 302f passes through continuous bore 350 and passes out of bone anchor 300f into the bone through one or more proximal aperture 352, distal aperture 354 or tip aperture 356 which communicate with continuous bore 350. The location and number of apertures can be varied to achieve a desired distribution of bone cement. In embodiments, only the proximal aperture 352, or the distal aperture 354 or the tip aperture 356 are present. For example, where the continuous bore 350 is to be used only for insertion of a guidewire or other tool, only tip aperture 356 is required to allow the guidewire to extend from tip 304f.

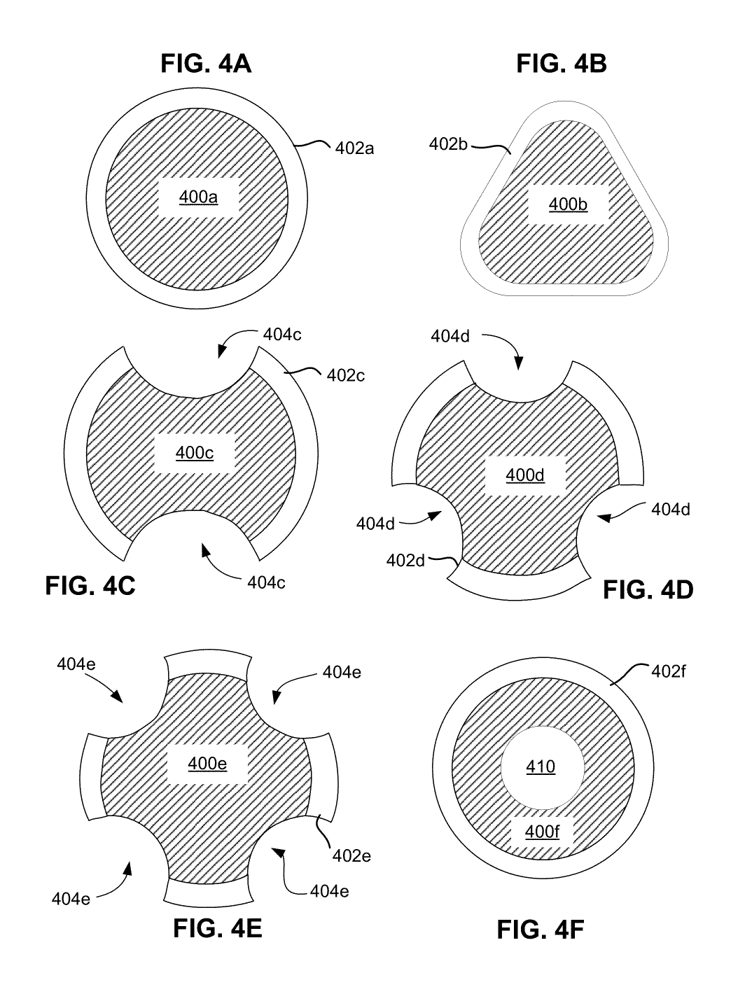

FIG. 3G shows a perspective view of a bone anchor 300g according to an alternative embodiment of the present invention. Bone anchor 300g includes a head 302g, at the proximal end and a tip 304g at the distal end. A shaft 306g extends between head 302g and tip 304g and includes a proximal shaft 320g and a distal shaft 340g. Proximal shaft 320g bears on its outside surface a single proximal thread 322g. Distal shaft 340g bears on its outside surface first and second distal threads 342g. First and second distal threads 342g merge together and connect to single proximal thread 322g at the transition 346g between the distal shaft 340g and proximal shaft 320g. The proximal thread 322g has a thread depth and threadform suitable for engaging bone and the proximal thread pitch 312g on the proximal shaft 320g is equal to the lead 310g. The distal threads 342g have a thread depth and threadform suitable for engaging bone cement, and the distal thread pitch 314g on the distal shaft 340g is half of the proximal thread pitch 312g on the proximal shaft 320g, and, thus, equal to half of the lead 310g. In the alternative embodiment shown in FIG. 3G, a longitudinal groove 360 has been cut into the distal shaft 340g and distal threads 342g. Although a single groove 360 is shown, a number of grooves, for example, 1, 2, 3 or 4, grooves 360 can be spaced around the distal shaft 340g. The distal threads 342g are segmented by groove 360, however, the segments are aligned as if part of a contiguous thread. Because the thread in this embodiment only intermittently engages the bore around the perimeter of the shaft, the thread places less stress on the bore reducing the chance of fracture. Moreover, any bone cement which is displaced during implantation can collect in a void formed by the groove 360 rather than being compressed and possibly causing facture. (See, also FIGS. 4C-4E and accompanying text.)

FIG. 3H shows a perspective view of a bone anchor 300h according to an alternative embodiment of the present invention. Bone anchor 300h includes a head 302h (which is in this case a polyaxial head), at the proximal end and a tip 304h at the distal end. A shaft 306h extends between head 302h and tip 304h and includes a proximal shaft 320h and a distal shaft 340h. Proximal shaft 320h bears on its outside surface a single proximal thread 322h. Distal shaft 340h bears on its outside surface first and second distal threads 342h. First and second distal threads 342h merge together and connect to single proximal thread 322h at the transition 346h between the distal shaft 340h and proximal shaft 320h. The proximal thread 322h has a thread depth and threadform suitable for engaging bone and the proximal thread pitch 312h on the proximal shaft 320h is equal to the lead 310h. The distal threads 342h have a thread depth and threadform suitable for engaging bone cement, and the distal thread pitch 314h on the distal shaft 340h is half of the proximal thread pitch 312h on the proximal shaft 320h, and, thus, equal to half of the lead 310h. In the alternative embodiment shown in FIG. 3H, the distal shaft 340h has the same major diameter as the proximal shaft 320h along most of its length. Adjacent tip 304h, the distal shaft 340h tapers rapidly to form a conical segment 370. Note also, that a self-tapping groove 372 is made in the surface of distal shaft 340h and distal threads 342h in the region of conical segment 370. Conical segment 370 and self tapping groove 372 serve to facilitate implantation of bone anchor 300h without fracturing bone cement. A self tapping groove 372 or conical segment 370 may be incorporated into any of the other distal shaft designs described herein.

### Variations of Bone Anchor Shaft Cross-Section

In the Figures, the shafts of the bone anchors are illustrated as having a generally circular solid cross-section in a plane perpendicular to the longitudinal axis of the shaft. Thus, the shafts are shown as generally cylindrical or conical/truncated conical. FIG. 4A schematically represents the cross-section of a circular shaft 400a having one or more threads 402a. For clarity of shaft shape, the position of thread(s) 402a is shown schematically as the projection of the threads into the plane of the section—the section of the thread is not shown. Although this is the most commonly used cross-section for a bone screw, alternative cross-sections are used in some embodiments. FIGS. 4B-4F illustrate alternative shaft cross-sections which can be utilized in place of the circular cross-section shown in any of the shaft embodiments illustrated herein. The proximal and distal shafts may have the same or different of the cross-sections shown in FIGS. 4A-4F. In particular embodiments, the proximal shaft of the bone anchor has the cross-section shown in FIG. 4A, and the distal shaft has one of the cross-sections illustrated in FIGS. 4B-4E.

FIG. 4B illustrates a shaft 400b having a tri-lobular or generally triangular shape. The thread 402b is illustrated as also triangular. The thread only engages the wall of the bore into which it is placed at the maximum radius from the center of the shaft. Essentially the thread will only engage the bore at the tips of the triangle. In alternative embodiments, the thread need not be continuous along the walls of the shaft. Because the thread 402b only intermittently engages the bore around the perimeter of the shaft 400b, the force required to drive the bone anchor is reduced thereby placing less stress on the bore reducing the chance of fracture. Moreover, any bone cement which is displaced by the thread 402b during implantation can collect in voids between the apices of the triangle rather than being compressed and possibly causing facture. Furthermore, cold flow of PMMA into the void after implantation can serve to lock in the bone anchor—reducing the chance that it will back out of the bore.

FIG. 4C illustrates a shaft 400c have generally circular shape from which two segments/grooves 404c have been cut. The thread 402c is generally circular. The thread only engages the wall of the bore into which it is placed at the maximum radius from the center of the shaft. The thread has been removed between the apices during formation of the two grooves 404c. Consequently, the thread is segmented along the perimeter of the shaft although the segments are aligned as if the thread remained contiguous. Similarly, FIG. 4D illustrates a generally circular shaft 400d having a thread 402d and in which three grooves 404d have been cut thereby segmenting thread 402d into three segments. Likewise FIG. 4E illustrates a generally circular shaft 400e having a thread 402e and in which four grooves 404e have been cut thereby segmenting thread 402e into four segments. Because the thread in these embodiments only intermittently engages the bore around the perimeter of the shaft, the force required to drive the bone anchor is reduced thereby placing less stress on the bore reducing the chance of fracture. Moreover, any bone cement which is displaced during implantation can collect in voids formed by the grooves rather than being compressed and possibly causing facture. Furthermore, cold flow of bone cement into the void(s) after implantation can serve to lock in the bone anchor—reducing the chance that it will back out of the bore.

FIG. 4F similarly represents the cross-section of a circular shaft 400f having one or more threads 402f wherein the shaft is cannulated and has a central bore 410 (as previously described). A central bore 410 may similarly be provided in each of the other shaft cross-sections shown in FIGS. 4A-4E if desired for receiving a guidewire or tool, or for the injection of bone cement.

### Variations Of Bone Anchor Tip

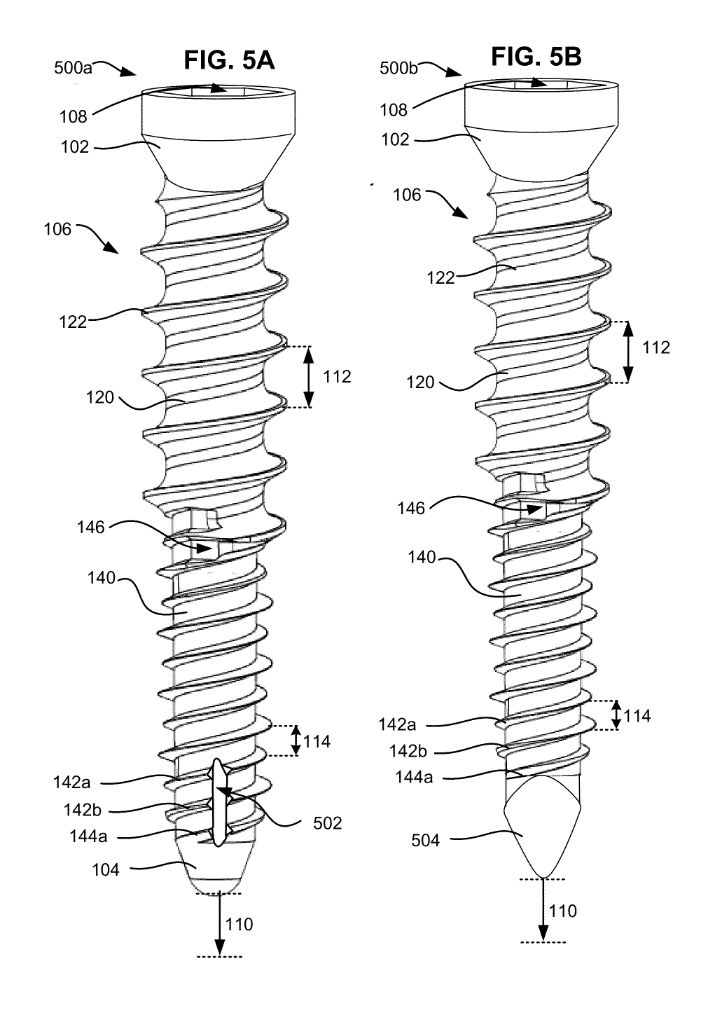

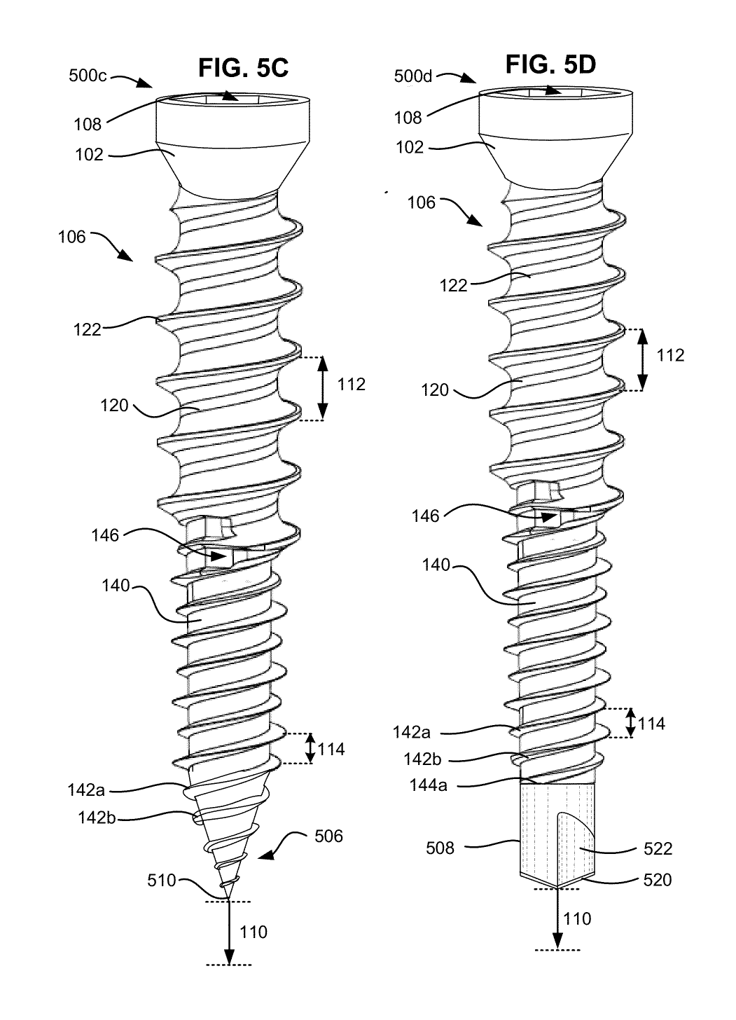

The inventive bone anchor shaft described herein is useful for anchoring a variety of orthopedic implants in the situation where a bone screw must be implanted in a bone which has been previously treated with bone cement, and, therefore, contains hard bone cement material. Although a blunt tip is shown in many of the figures, in alternative embodiments a different bone anchor tip suitable for a particular application may be used in combination with any one of the disclosed shafts including, but not limited to: a self-tapping tip; rounded tip; blunt tip; blunt self-tapping tip; trocar tip; tapered tip; or corkscrew tip. FIGS. 5A-5B show alternative tip embodiments which can replace the tips shown in the otherwise disclosed embodiments.

FIG. 5A, illustrates a variation 500a of bone anchor 100 of FIGS. 1A-1F having a self tapping tip. A blunt tip, as shown in FIG. 1A, allows for good accuracy of implantation in a pre-drilled bore. However, the blunt tip displaces bone cement cut by the threads in the distal bore. As shown in FIG. 5A, the bone anchor 500a can be provided with one or more flutes 502 cut into the distal threads 142a, 142b adjacent the tip 104 to allow cuttings created during the formation of threads in the bore to escape. The provision of flutes 502 prevents or reduces the accumulation of cuttings ahead of the screw tip which might lead to fracture of the bone cement. The sharpness, number, and geometry of flute(s) 502 are selected to be effective to avoid facture of the bone cement material. (See, also FIGS. 3G, 3H, 4B-4F and accompanying text.)

FIG. 5B illustrates a variation 500b of bone anchor 100 of FIGS. 1A-1F having a trocar tip 504. Trocar tip 504 is sharper and more tapered than rounded tip 104 of FIG. 1A. Trocar tip 504 is, in an alternative embodiment, provided with one or more flutes.

FIG. 5C illustrates a variation 500c of bone anchor 100 of FIGS. 1A-1F having a sharp tapered tip 506. Tip 506 facilitates implantation of bone anchor 500c into bone cement. Tip 506 tapers rapidly from the minor diameter of the distal shaft 140 to a sharp point 510. Sharp point 510 enables sharp tapered tip 506 to penetrate bone cement during implantation.

FIG. 5D illustrates a variation 500d of bone anchor 100 of FIGS. 1A-1F having a drill tip 508. Drill tip 508 facilitates implantation of bone anchor 500d into bone cement. The drill tip 508 can form the distal bore simultaneously with implantation of bone anchor 500d. Alternatively, a pilot bore is predrilled and drill tip 508 enlarges the bore during implantation of bone anchor 500d. Drill tip 508 includes one or more sharp cutting lips 520 and one or more flutes 522. During operation lips 520 cut into bone cement thereby forming the distal bore in all or in part.

### Variations of Bone Anchor Head

The bone anchor shaft described herein is useful for anchoring a variety of orthopedic implants in the situation where a bone screw must be implanted in a bone which has been previously treated with bone cement, and, therefore, contains hard bone cement material. The head of the bone anchor is selected to be suitable for the secure connection of a spinal prosthesis component, and, thus, the spinal prosthesis to the bone anchor whereby the spinal prosthesis is effectively secured to the bone in which the bone anchor is implanted. Although a simple head is shown in many of the figures, in alternative embodiments a different bone anchor head suitable for a particular application may be used in combination with any one of the disclosed shafts. In embodiments, the bone anchor head is selected from: Steffee heads; hex heads; hex socket heads; Torx heads; breakaway heads; fixed heads; polyaxial heads, pedicle screw heads; angled heads; dynamic bone anchor heads; and other heads desired to be securely mounted to a bone containing hardened bone cement. In principle, any conventional or future-developed bone anchor head can be combined with the shaft of this invention where it is desired to secure the head to a bone which has been previously treated with bone cement.

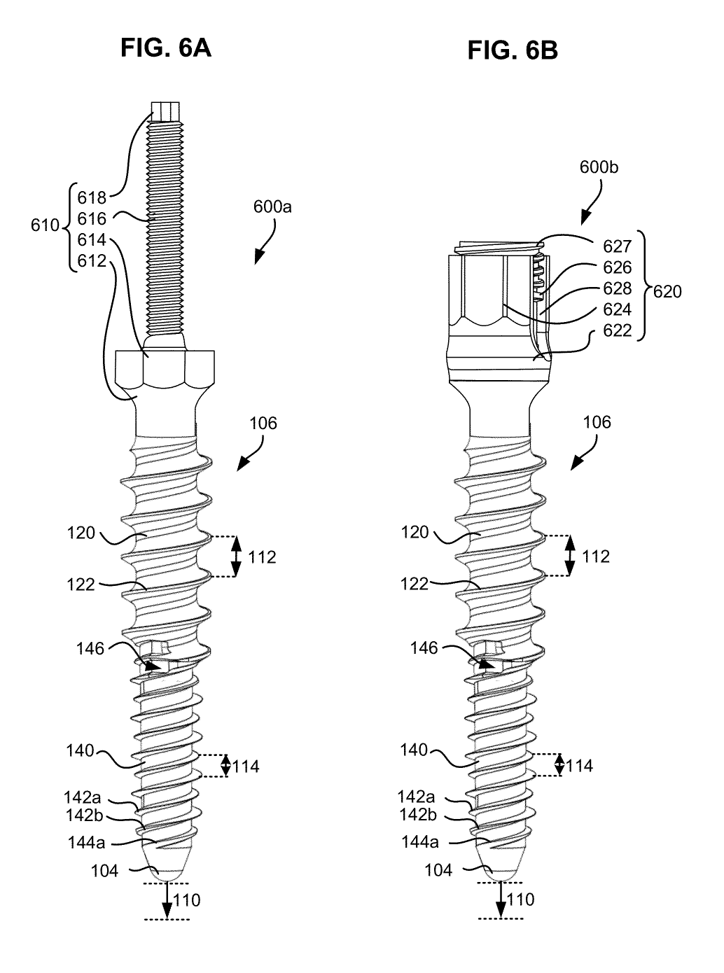

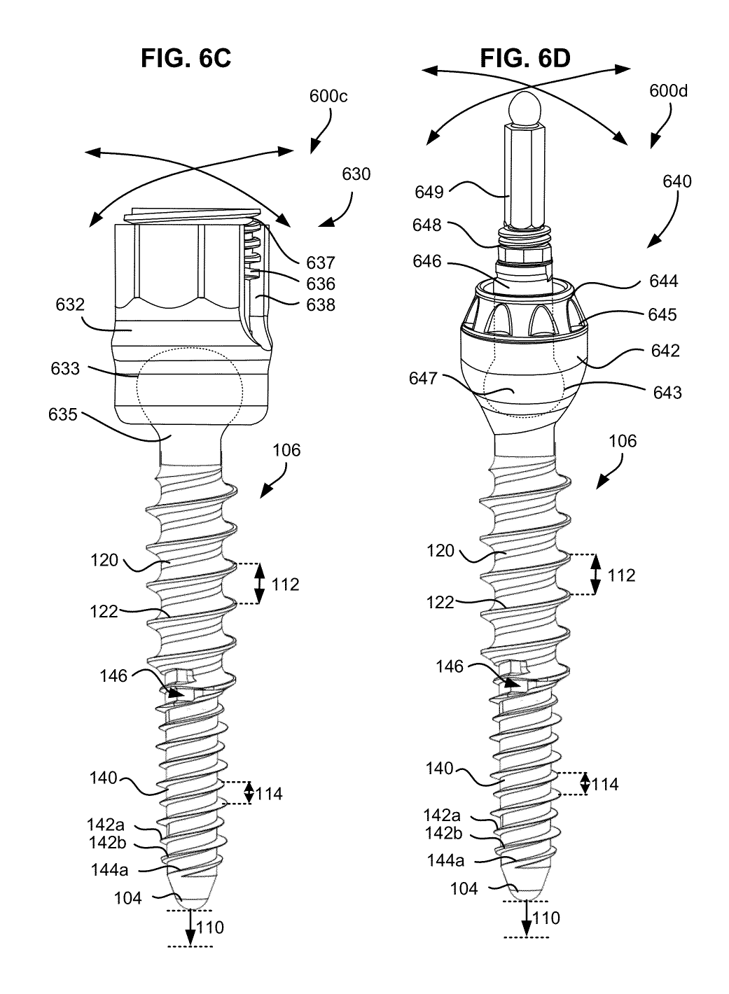

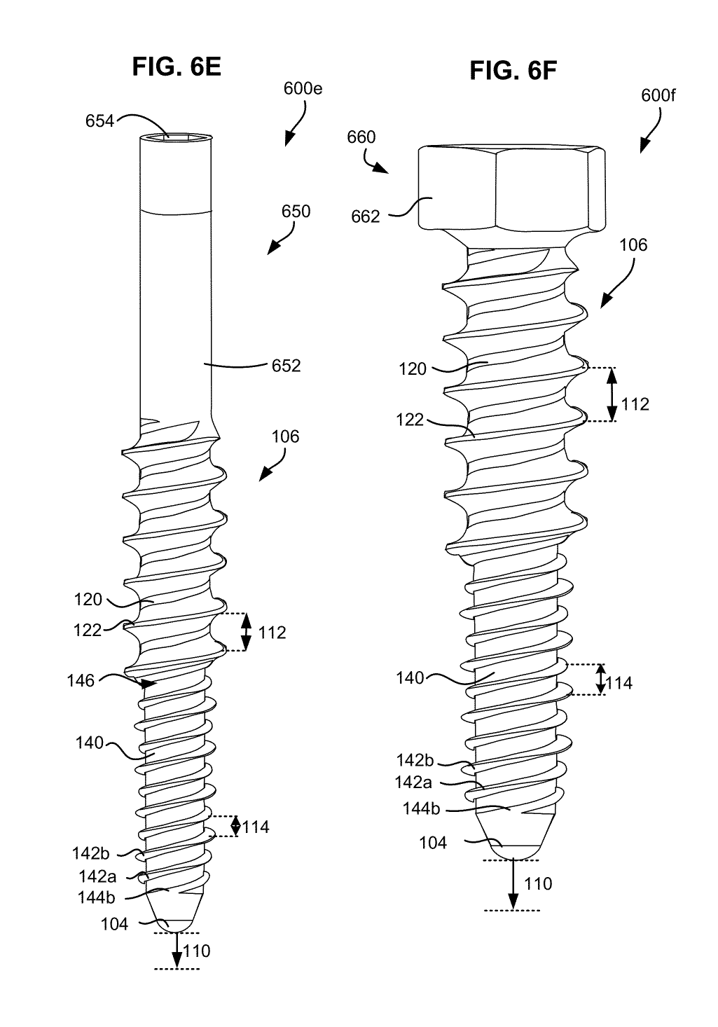

FIGS. 6A-6F show alternative heads which can replace the heads shown in the otherwise disclosed embodiments. FIG. 6A, illustrates a variation 600a of bone anchor 100 of FIGS. 1A-1F having a Steffee type head 610 suitable for mounting a plate or other spinal implant component to a bone. As shown in FIG. 6A, head 610 includes a base 612 having a hexagonal external surface 614 which can be engaged by a wrench for turning bone anchor 600a during implantation. A threaded post 616 extends proximally from base 612. At the proximal end of threaded post 616 is a hex end 618 which can also be engaged by a wrench. In use, bone anchor 600a is implanted in a bone, and a plate (not shown) is, thereafter, secured to threaded post 616 with one or more nuts (not shown).

FIG. 6B, illustrates a variation 600b of bone anchor 100 of FIGS. 1A-1F having a pedicle screw head 620 suitable for mounting a spinal rod or other spinal implant component to a bone. As shown in FIG. 6B, head 620 includes a body 622 having a hexagonal external surface 624 which can be engaged by a wrench for turning bone anchor 600b during implantation. Body 622 has a fixed relationship to shaft 106. A threaded bore 626 extends into body 622. Threaded bore 626 receives a threaded set screw 627. Threaded bore 626 is slotted 628 such that a rod can be inserted across threaded bore 626. In use, bone anchor 600b is implanted in a bone, and a rod (not shown) is, thereafter, inserted through slots 628 across threaded bore 626. Set screw 627 is then tightened to secure the rod to head 620.

FIG. 6C, illustrates a variation 600c of bone anchor 100 of FIGS. 1A-1F having a polyaxial pedicle screw head 630 suitable for mounting a spinal rod or other spinal implant component at an adjustable angle to a bone. As shown in FIG. 6C, head 630 includes a body 632. Body 632 has a socket 633 which is mounted to a coupling 635 formed at the end of shaft 106. Socket 633 is mounted to coupling 635 such that body 632 can be arranged at a variable angle and/or rotation relative to shaft 106. A threaded bore 636 extends into body 632. Threaded bore 636 receives a threaded set screw 637. Threaded bore 636 is slotted 638 such that a rod can be inserted across threaded bore 636. In use, bone anchor 600c is implanted in a bone—in some embodiments coupling 635 includes a hex socket which can be engaged by a wrench for turning bone anchor 600c during implantation. After implanting bone anchor 600c, a rod (not shown) is, thereafter, inserted through slots 638 across threaded bore 636. Set screw 637 is then tightened to secure the rod to head 630 and lock the angle of body 632 relative to shaft 106. Polyaxial screw head 630 is shown in simplified form and may include one or more elements not shown. A wide variety of polyaxial heads is known in the art and is suitable for combining with shaft 106. The term polyaxial head is meant to encompass all of the various polyaxial heads known in the art.

FIG. 6D illustrates a variation 600d of bone anchor 100 of FIGS. 1A-1F having a dynamic head 640 suitable for mounting a spinal rod or other spinal implant component to a bone in a manner which allows motion preservation and load sharing. As shown in FIG. 6D, dynamic head 640 includes a body 642. Body 642 has a socket 643 and a cap 644. Body 642 has surface feature 645 which can be engaged by a wrench for turning bone anchor 600d during implantation. A deflectable post 646 includes a distal coupling 647 received in socket 643 and extending through cap 644. Coupling 647 is mounted and retained in socket 643 such that deflectable post 646 can deflect through a range of angles and/or rotate relative to shaft 106 even after a spinal rod or other spinal implant component is mounted to deflectable post 646. Movement of deflectable post 646 is constrained by contact with cap 644. Deflectable post 646 includes a threaded mount 648 to which a spinal rod or other spinal implant component can be secured with a nut without locking the angle of deflectable post 646 relative to shaft 106. Threaded mount 648 includes one or more features 649 (e.g. a hex extension and/or hex socket) which allow deflectable post 646 to be engaged by a wrench during the securing of a rod or other spinal implant component to deflectable post 646. In use, bone anchor 600d is implanted in a bone. After implanting bone anchor 600d, a rod (not shown) is thereafter inserted over deflectable post 646 and secured to threaded mount 648 with a nut (not shown). Note that dynamic head 640 is designed such that it secures the rod or other spinal component to the shaft 106 while still permitting constrained movement of the rod or other spinal component relative to the shaft 106 in a manner which allows motion preservation and load sharing. A wide variety of dynamic heads is taught in U.S. patent application Ser. No. 13/352,882 entitled “Low Profile Spinal Prosthesis Incorporating A Bone Anchor Having A Deflectable Post And A Compound Spinal Rod” filed Jan. 18, 2012, which is hereby incorporated by reference in its entirety. These dynamic heads are suitable for combining with shaft 106. The term “dynamic stabilization head” is meant to encompass all of the various dynamic heads disclosed in U.S. patent application Ser. No. 13/352,882.

FIG. 6E, illustrates a variation 600e of bone anchor 100 of FIGS. 1A-1F having a post type head 650 suitable for mounting a rod or other spinal implant component to a bone. As shown in FIG. 6E, head 650 includes a post 652. Post 652 has at its proximal end a hexagonal socket 654 which can be engaged by a wrench for turning bone anchor 600e during implantation. In use, bone anchor 600e is implanted in a bone, and a rod is thereafter secured to post 652 with a coupling (not shown).

FIG. 6F, illustrates a variation 600f of bone anchor 100 of FIGS. 1A-1F having a hex type head 660 suitable for mounting a plate or other spinal implant component to a bone. As shown in FIG. 6F, head 660 has a hexagonal exterior surface 662 which can be engaged by a wrench for turning bone anchor 600f during implantation.

### Dynamic Bone Anchor

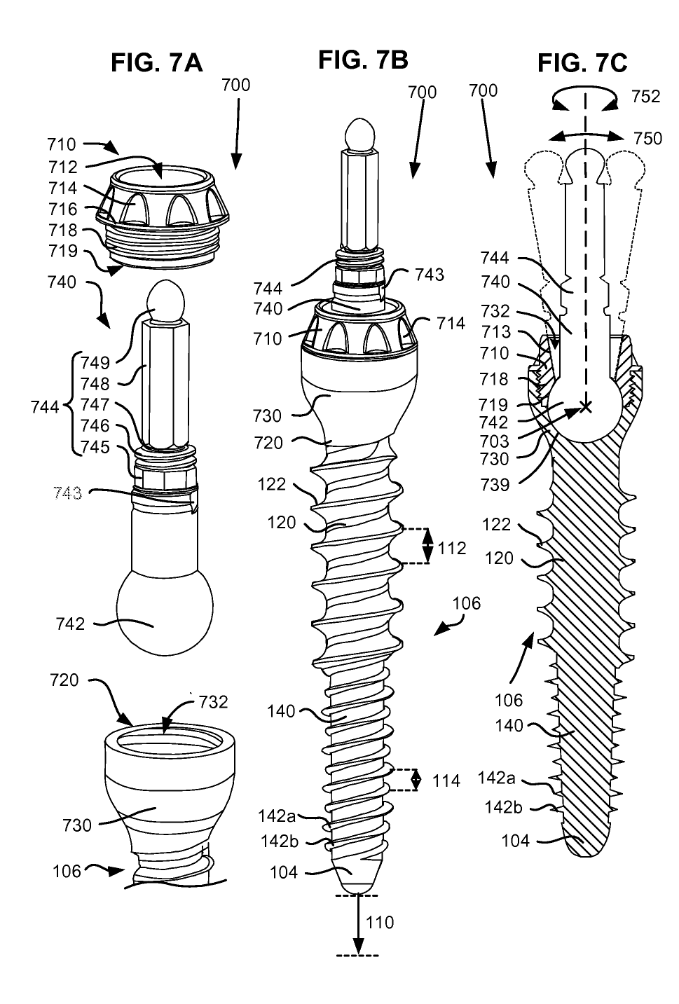

FIGS. 7A-7C illustrate a dynamic bone anchor 700 incorporating the shaft of the bone anchor 100 of FIGS. 1A-1F in conjunction with one embodiment of a dynamic stabilization head. FIG. 7A shows an exploded view of bone anchor 700. FIG. 7B shows a perspective view of bone anchor 700, as assembled. FIG. 7C shows a sectional view of bone anchor 700. Referring first to FIG. 7A, bone anchor 700 includes, in this embodiment, three components: bone screw 720, deflectable post 740, and cap 710. Bone screw 720 comprises a threaded shaft 106 (which is the same as shaft 106 described in FIGS. 1A-1F) with a housing 730 at one end. Housing 730 may, in some embodiments, be cylindrical, and, is, in some embodiments, provided with splines/flutes. Housing 730 is preferably formed in one piece with threaded shaft 106. Housing 730 has a cavity 732 oriented along the axis of threaded shaft 106. Cavity 706 is open at the proximal end of housing 730 and is configured to receive deflectable post 740.

In a preferred embodiment, deflectable post 740 is a titanium post 5 mm in diameter. Deflectable post 740 has a retainer 742 at one end. At the other end of deflectable post 740, is a mount 744. Retainer 742 is a ball-shaped or spherical structure in order to form part of a linkage connecting deflectable post 740 to bone screw 720. Mount 744 is a low profile mount configured to connect deflectable post 740 to a spinal rod (not shown). Mount 744 comprises a threaded cylinder 746 to which the vertical rod component may be secured. Mount 744, in some embodiments, also comprises a polygonal section 745 to prevent rotation of a component relative to mount 744.

Mount 744 includes a male hex extension 748 which may be engaged by a tool to hold stationary mount 744 during attachment to a vertical rod. At the proximal end of male hex extension 748 is a nipple 749 for securing male hex extension 748 into a tool. Hex extension 748 is a breakaway component. Between hex extension 748 and threaded cylinder 746 is a groove 747. Groove 747 reduces the diameter of deflectable post 740 such that hex extension 748 breaks away from threaded cylinder 746 when a desired level of torque is reached during attachment of a vertical rod. The breakaway torque is determined by the diameter of remaining material and the material properties. In a preferred embodiment, the breakaway torque is approximately 30 foot pounds. Thus, hex extension 748 breaks away during implantation and is removed. Nipple 749 is engaged by the tool in order to remove hex extension 748. Deflectable post 740 is also provided with flats 743 immediately adjacent mount 744. Flats 743 allow deflectable post 740 to be engaged by a tool after hex extension 748 has been removed.

Referring again to FIG. 7A, a cap 710 is designed to perform multiple functions including securing retainer 742 in cavity 732 of bone screw 720. Cap 710 has a central aperture 712 for receiving deflectable post 740. In the embodiment of FIG. 7A, cap 710 has surface features 714, for example, splines or flutes, adapted for engagement by an implantation tool or mounting a component, e.g. an offset connector. Surface features 714 may be, for example, engaged by a driver that mates with surface features 714 for implanting bone anchor 700 in a bone. As shown in FIG. 7A, cap 710 comprises a cylindrical shield section 718 connected to a collar section 716. Shield section 718 is designed to mate with cavity 732 of housing 730. Shield section 718 is threaded adjacent collar section 716 in order to engage threads at the proximal end of cavity 732 of housing 730. The distal end of shield section 718 comprises a flange 719 for securing retainer 742 within cavity 732 of housing 730.

Bone anchor 700 is assembled prior to implantation in a patient. FIG. 7B shows a perspective view of bone anchor 700 as assembled. When assembled, deflectable post 740 is positioned through cap 710. Cap 710 is then secured to the threaded end of cavity 732 (see FIGS. 7A and 7C) of housing 730 of bone screw 720. Cap 710 has surface features 714 for engagement by a wrench to allow cap 710 to be tightened to housing 730. For example, cap 710 may be hexagonal or octagonal in shape or may have splines and/or flutes and/or other registration elements. Cap 710 may alternatively, or additionally, be laser welded to housing 730 after installation. Cap 710 secures deflectable post 740 within cavity 732 of bone screw 720. Deflectable post 740 extends out of housing 730 and cap 710 such that mount 744 is accessible for connection to a vertical rod. Bone anchor 700 is implanted in a bone in the configuration shown in FIG. 7B and prior to attachment of a vertical rod or other spinal rod. A special tool may be used to engage the surface features 714 of cap 710 during implantation of bone anchor 700 into a bone.

As previously described, threaded shaft 106 includes a tip 104 at the distal end. Shaft 106 extends between housing (head) 730 and tip 104 and includes a proximal shaft 120 and a distal shaft 140. Proximal shaft 120 bears on its outside surface a single proximal thread 122. Distal shaft 140 bears on its outside surface first and second distal threads 142a, 142b. First and second distal threads 142a, 142b merge together and connect to single proximal thread 122 at the transition between the distal shaft 140 and proximal shaft 120. The proximal thread 122 has a thread depth and threadform suitable for engaging bone and the proximal thread pitch 112 on the proximal shaft 120 is equal to the lead 110. The distal threads 142a, 142b have a thread depth and threadform suitable for engaging hardened bone cement, and the distal thread pitch 114 on the distal shaft 140 is half of the proximal thread pitch 112 on the proximal shaft 120, and, thus, equal to half of the lead 110. In conjunction with threaded shaft 106, dynamic bone anchor 700 can be utilized to provide dynamic stabilization of a vertebra previously treated with bone cement.

FIG. 7C shows a sectional view of a bone anchor 700. Retainer 742 fits into a hemispherical pocket 739 in the bottom of cavity 732 of housing 730. The bottom edge of cap 710 includes the curved flange 719 which secures ball-shaped retainer 742 within hemispherical pocket 739 while allowing ball-shaped retainer 742 to pivot and rotate. Accordingly, in this embodiment, a ball-joint is formed. FIG. 7C also illustrates deflection of deflectable post 740 shown in dashed lines. Applying a force to mount 744 causes deflection of deflectable post 740 of bone anchor 700. Deflectable post 740 pivots about a pivot point 703 indicated by an X. Deflectable post 740 may pivot about pivot point 703 in any direction, as shown by arrow 750. Concurrently or alternatively, deflectable post 740 can rotate, as shown by arrow 752, about the long axis of deflectable post 740 (which also passes through pivot point 703). In this embodiment, pivot point 703 is located at the center of ball-shaped retainer 742.

Dynamic bone anchor 700 is designed such that deflectable post 740 remains deflectable after the mounting of a spinal rod or other spinal implant to deflectable post 740. In this way, dynamic bone anchor stabilizes the spine while still permitting relative movement of vertebrae of the spine within constraints imposed by the limits of deflection of deflectable post 740. In a preferred embodiment, deflectable post 740 may deflect from 0.5 mm to 2 mm in any direction before making contact with limit surface 713. More preferably, deflectable post 740 may deflect approximately 1 mm before making contact with limit surface 713. After a fixed amount of deflection, deflectable post 740 comes into contact with limit surface 713 of cap 710. Limit surface 713 is oriented such that when deflectable post 740 makes contact with limit surface 713, the contact is distributed over an area to reduce stress on deflectable post 740. In this embodiment, the deflectable post 740 contacts the entire sloping side of the conically-shaped limit surface 713. In another embodiment, the deflectable post may only contact a limit ring that is located distally from the flange 719 of cap 710. After deflectable post 740 comes into contact with limit surface 713, further deflection requires deformation (bending) of deflectable post 740.

The configuration and materials of the dynamic head may be selected to create a deflection assembly having stiffness/deflection characteristics suitable for the needs of a patient. By selecting appropriate dimensions and materials, the deflection characteristics of the deflectable post can be configured to approach the natural dynamic motion of the spine of a particular patient, while giving dynamic support to the spine in that region. It is contemplated, for example, that the spinal prosthesis utilizing the bone anchor having a dynamic head can be made in stiffness that can replicate a 70% range of motion and flexibility of the natural intact spine, a 50% range of motion and flexibility of the natural intact spine and a 30% range of motion and flexibility of the natural intact spine.

In alternative embodiments, a compliant member/sleeve/ring can be added to the bone anchor 700 positioned within housing 730, cap 710, and/or deflectable post 740. The compliant member is positioned such that it is compressed by deflection of deflectable post 740 away from alignment with the longitudinal axis of shaft 106. As a result of such compression, the compliant member exerts a restoring force upon deflectable post 740 pushing it back into alignment with the longitudinal axis of shaft 106. The compliant member can be, for example, a metal, superelastic, nitinol, or polymer member. The material of the compliant member/sleeve/ring is, in some embodiments, a biocompatible and implantable polymer having the desired deformation characteristics. The sleeve may, for example, be made from PEEK or a polycarbonate urethane (PCU) such as Bionate®. If the sleeve is comprised of Bionate®, a polycarbonate urethane or other hydrophilic polymer, the sleeve can also act as a fluid-lubricated bearing for rotation of the deflectable post relative to the longitudinal axis of the deflectable post.

Movement of the deflectable post relative to the bone anchor provides load sharing and dynamic stabilization properties to the dynamic stabilization assembly. As described above, deflection of the deflectable post deforms the material of the sleeve. The characteristics of the material of the sleeve in combination with the dimensions of the components of the deflection rod assembly affect the force-deflection curve of the deflection rod. By changing the dimensions of the deflectable post, sleeve and the shield, the deflection characteristics of the deflection rod assembly can be changed. The stiffness of components of the deflection rod assembly can be, for example, increased by increasing the diameter of the deflectable post and/or by decreasing the diameter of the inner surface of the shield. Additionally, decreasing the diameter of the deflectable post will decrease the stiffness of the deflection rod assembly while decreasing the diameter of the post and/or by increasing the diameter of the inner surface of the shield will decrease the stiffness of the deflection rod. Alternatively and/or additionally, changing the materials which comprise the components of the deflection rod assembly can also affect the stiffness and range of motion of the deflection rod. For example, making the sleeve out of stiffer and/or harder material reduces deflection of the deflectable post.

Particular embodiments of dynamic bone anchors, deflectable posts with and without compliant members/sleeves/rings, and dynamic spinal stabilization systems are disclosed in U.S. patent application Ser. No. 13/352,882 entitled “Low Profile Spinal Prosthesis Incorporating A Bone Anchor Having A Deflectable Post And A Compound Spinal Rod” filed Jan. 18, 2012, which is hereby incorporated by reference in its entirety. The embodiments of bone anchor shafts and tips and installation tools and methods described in the present patent application can be utilized with any of the bone anchor embodiments disclosed in patent application Ser. No. 13/352,882 by replacing/modifying the bone anchor shafts and tips and installation tools and methods disclosed in patent application Ser. No. 13/352,882 with those described in the present patent application for use in situations where implantation is required in a vertebra including hardened bone cement.

### Alternative Bone Anchor Implantation Tools

As described with respect to FIGS. 2E, 2F, the distal bore 234 is, in some procedures, created by heating of the bone cement 214 before or during implantation of a bone anchor. To form distal bore 234 during implantation of a bone anchor, the bone anchor is provided with means for melting the bone cement during implantation. In one method, a heated probe inserted through a cannulated bone anchor is used to melt the PMMA adjacent the tip of the bone anchor. The melted PMMA can be displaced or removed during insertion of the bone anchor. Alternatively, the tip of the bone anchor itself is heated rather than a separate probe. The probe or anchor tip can be heated electrically, ultrasonically or using electromagnetic radiation, for example, an infrared laser. Alternatively, the distal bore is created using a mechanical tool such as a rotating burr inserted through a cannulated bone anchor that mechanically heats the PMMA above its melting temperature during implantation of the bone anchor. Alternatively, the distal bore is created using a drill and then the bone cement surrounding the distal bore is heat treated before or during bone anchor implantation to fuse any fractures that may have been formed during the cutting of the distal bore.

FIG. 8A illustrates a cannulated bone anchor 300f as previously described with respect to FIG. 3F in conjunction with a heated probe 840 which includes a shaft 842 and heated tip 844. A power/temperature controller 846 is coupled to heated tip 844 through shaft 842. The power/temperature controller 846 provides one of electrical, ultrasonic or electromagnetic energy to heat heated tip 844. Heated probe 840 is inserted through a channel 802 in a wrench 800 having a head 804 adapted to engage the head 302f of bone anchor 300f in order to turn bone anchor 300f during implantation. Heated probe 840 may be fixed in wrench 800 or removable. Shaft 842 extends beyond head 804 through continuous bore 350 and out of tip aperture 356 of bone anchor 300f. Shaft 842 has a length selected such that heated tip 844 protrudes beyond the tip 304f of bone anchor 300f.

In use, the physician operates power/temperature controller 846 to raise the temperature of heated tip 844 above the glass transition temperature of bone cement. The physician utilizes wrench 800 to drive bone anchor 300f into the vertebra. Heated tip 844 heats the bone cement adjacent the tip 304f of bone anchor 300f. Melted bone cement flows away from heated tip 844 as heated tip 844 is introduced with bone anchor 300f creating the distal bore simultaneous with implantation. Heated probe 840 and/or bone anchor 300f are, in some embodiments, provided with channels and/or grooves which allow melted bone cement to flow towards the proximal bore 232 during implantation. When the bone anchor has been implanted to its desired position in the bone, heated probe 840 and wrench 800 are removed. In this embodiment the distal bore is formed simultaneously with the implantation of the bone anchor.

FIG. 8B illustrates a cannulated bone anchor 300f similar to that previously described with respect to FIG. 3F in conjunction with a heating system 850 for heating the tip 304f of bone anchor 300f. As shown in FIG. 8B, continuous bore 350 extends from head 302f but terminates just before the surface of tip 304f. Bone anchor 300f has, in this embodiment, no tip aperture. A power/temperature controller 856 is coupled to tip 854 through fiber 852. The power/temperature controller 856 provides one of electrical, ultrasonic or electromagnetic energy to heat the tip 304f of bone anchor 300f. In some embodiments, fiber 852 is inserted through a channel 802 in a wrench 800 having a head 804 adapted to engage the head 302f of bone anchor 300f in order to turn bone anchor 300f during implantation. Fiber 852 may be fixed in wrench 800 or removable. Fiber 852 extends beyond head 804 through continuous bore 350 of bone anchor 300f. Fiber 852 has a length selected such that tip 854 is just proximal of the distal end of continuous bore 350. Tip 854 is designed to deliver heat energy to tip 304f of bone anchor 300f thereby raising the temperature of tip 304f of bone anchor 300f.

In one embodiment fiber 852 is an optical fiber which transmits laser light from power/temperature controller 856 to tip 854. Tip 854 is designed to emit the laser light such that it is incident upon and heats the tip 304f of bone anchor 300f. Power/temperature controller 856 monitors tip temperature by assessing electromagnetic radiation returned through fiber 852. In this way, closed-loop temperature control of the tip 304f of bone anchor 300f can be achieved.

In use, the physician operates heating system 850 to raise the temperature of tip 304f above the glass transition temperature of bone cement. The physician utilizes wrench 800 to drive bone anchor 300f into the vertebra. Heated tip 304f heats the bone cement adjacent the tip 304f of bone anchor 300f. Melted bone cement flows away from heated tip 304f creating the distal bore simultaneous with implantation. Bone anchor 300f is, in some embodiments, provided with channels and/or grooves which allow melted bone cement to flow towards the proximal bore during implantation. When the bone anchor 300f has been implanted to its desired position in the bone, heating system 850 and wrench 800 are removed. In this embodiment, the distal bore is formed simultaneously with the implantation of the bone anchor. However, the heated tip of a bone anchor may also be used to anneal/fuse the walls of a pre-drilled/preformed distal bore.

FIG. 8C illustrates an alternative method for creating a distal bore in conjunction with implantation of a bone anchor. As before, the proximal bore is created using conventional methods for creating a bore in a vertebra, e.g. a blunt probe or drill. For example, a probe can be passed through the pedicle without excessive force until it contacts bone cement. When the probe contacts bone cement, it is removed and a rotary probe 860 is inserted through a channel 802 in a wrench 800 having a head 804 adapted to engage the head 302f of bone anchor 300f in order to turn bone anchor 300f during implantation. Rotary probe 860 includes a shaft 862 and burr tip 864. A driver 866 (for example, an electrical motor) is coupled to burr tip 864 through shaft 862. The driver 866 rotates shaft 862 and burr tip 864 at high speed. Rotary probe 860 may be fixed in wrench 800 or removable. Shaft 862 extends beyond head 804 through continuous bore 350 and out of tip aperture 356 of bone anchor 300f. Shaft 862 has a length selected such that burr tip 864 protrudes beyond the tip 304f of bone anchor 300f.

In use, the physician operates driver 866 to rotate the burr tip 864 at high speed. Friction between burr tip 864 and bone cement adjacent tip 304f raises the temperature of burr tip 864 and the bone cement above the glass transition temperature of the bone cement. The burr tip advances through the bone cement as the physician utilizes wrench 800 to rotate bone anchor 300f. The bone cement flows away from burr tip 864 as burr tip 864 is introduced, creating the distal bore simultaneous with implantation. Bone anchor 300f is, in some embodiments, provided with channels and/or grooves which allow melted bone cement to flow towards the proximal bore. When the bone anchor has been implanted in the desired position, rotary probe 860 and wrench 800 are removed. In this procedure burr tip 864 is used to melt the bone cement during implantation of the bone anchor thereby reducing the possibility of fracture. In this embodiment distal bore may be formed simultaneously with the implantation of the bone anchor.

FIG. 8D illustrates an alternative method for creating a distal bore in conjunction with implantation of a bone anchor. As before, the proximal bore is created using conventional methods for creating a bore in a vertebra, e.g. a blunt probe or drill. For example, a probe can be passed through the pedicle without excessive force until it contacts bone cement. When the probe contacts bone cement, it is removed and an ultrasonic probe 870 is inserted through a channel 802 in a wrench 800 having a head 804 adapted to engage the head 302f of bone anchor 300f in order to turn bone anchor 300f during implantation. Ultrasonic probe 870 includes a shaft 872 and ultrasonic tip 874. An ultrasonic transducer 876 is coupled to ultrasonic tip 874 through shaft 872. The ultrasonic transducer 876 provides ultrasound vibrations through shaft 872 to ultrasonic tip 874. Ultrasonic probe 870 may be fixed in wrench 800 or removable. Shaft 872 extends beyond head 804 through continuous bore 350 and out of tip aperture 356 of bone anchor 300f. Shaft 872 has a length selected such that ultrasonic tip 874 protrudes beyond the tip 304f of bone anchor 300f.

In use, the physician operates ultrasonic transducer 876 to vibrate the ultrasonic tip 874 at high frequency. High frequency vibration at the region of contact between ultrasonic tip 874 and bone cement adjacent tip 304f raises the temperature of ultrasonic tip 874 and the bone cement above the glass transition temperature of the bone cement. The ultrasonic tip 874 advances through the bone cement as the physician utilizes wrench 800 to rotate bone anchor 300f. The bone cement flows away from ultrasonic tip 874 as ultrasonic tip 874 is introduced—creating the distal bore simultaneous with implantation. Bone anchor 300f is, in some embodiments, provided with channels and/or grooves which allow melted bone cement to flow towards the proximal bore. When the bone anchor has been implanted in the desired position, ultrasonic probe 870 and wrench 800 are removed. In this procedure ultrasonic tip 874 is used to melt or soften the bone cement during implantation of the bone anchor thereby reducing the possibility of fracture. In this embodiment distal bore may be formed simultaneously with the implantation of the bone anchor.

### Heated Tip Bone Anchors

In alternative embodiments of the present invention, the bone anchor is provided with an integrated heated tip which is adapted to heat the bone cement adjacent the heated tip thereby softening and/or melting the bone cement to facilitate implantation of the bone anchor into bone cement without fracturing the bone cement. The heated tip can be utilized to entirely create the distal bore simultaneous with implantation. Alternatively, the distal bore (or a pilot bore) can be created in a preliminary step and the heated tip can be used to fuse and/or anneal the bone cement adjacent the bore preventing propagation of any fractures. The integrated heated tip can be, for example, a thermoelectrically heated tip, ultrasonically heated tip, or mechanically heated tip.

A thermoelectric heated tip converts electrical energy into heat energy which is then transmitted by conduction into the bone cement to soften and/or melt the bone cement adjacent the tip of the bone anchor during implantation. The thermoelectric tip can be blunt, tapered, or sharp, and can include the screw tip features previously disclosed including, but not limited to, one or more of threads, flutes, grooves, self tapping, drill, and a distal aperture. In preferred embodiments, two electrical conductors pass along the length of the bone anchor to the tip. The bone anchor shaft is used as one of the two conductors in some embodiments. The two conductors are coupled to a power supply which supplies electrical current to the thermoelectric tip which converts electrical energy into heat energy which heats the thermoelectric tip and the bone cement with which it is in contact. The thermoelectric tip may include one or more resistive heating elements which produce heat in response to an electrical current. The resistive heating elements can be formed from a material having a higher resistivity than the conductors and/or in a shape and size that has a higher resistance than the conductors such that heat is generated in the resistive elements rather than the conductors. If the material of the resistive element is not biocompatible the resistive elements are preferably encased or enclosed in a biocompatible material, for example, stainless steel or titanium. In preferred embodiments, the temperature of the thermoelectric tip is regulated such that it remains at a temperature suitable for softening and/or melting bone cement during implantation of the bone anchor without damaging surrounding tissues or burning the bone cement.

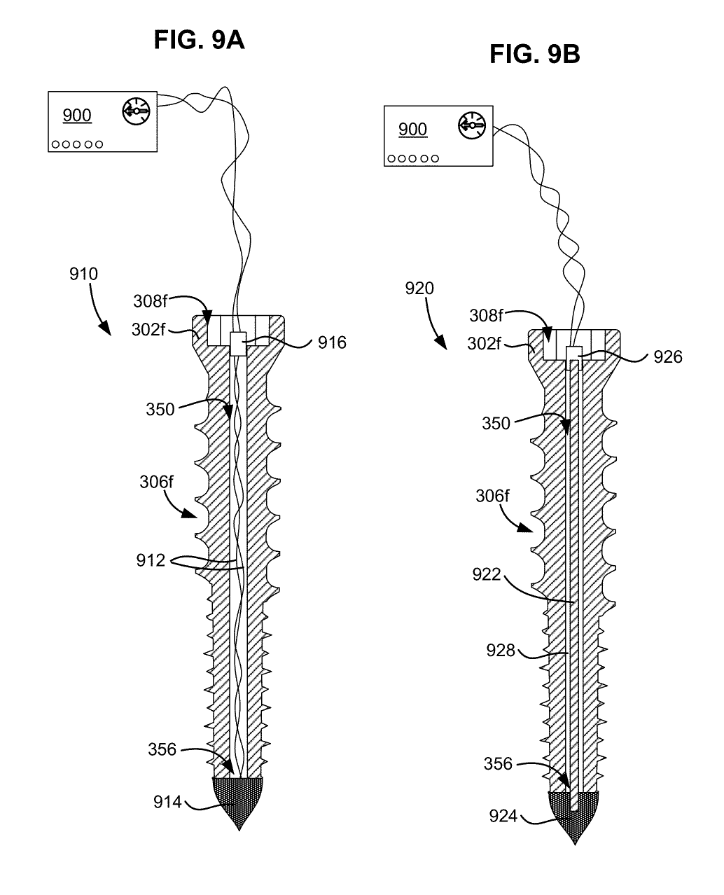

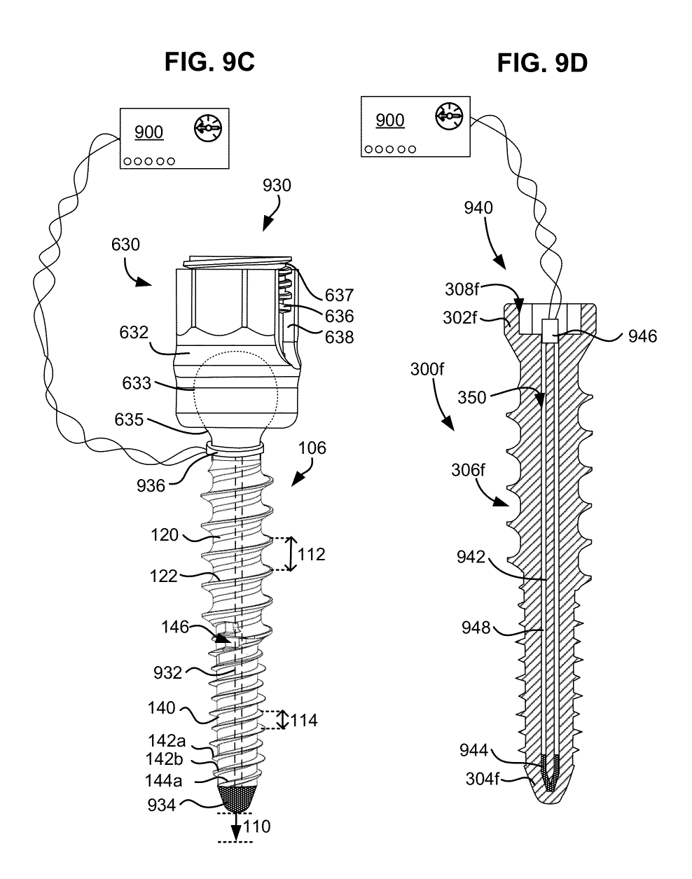

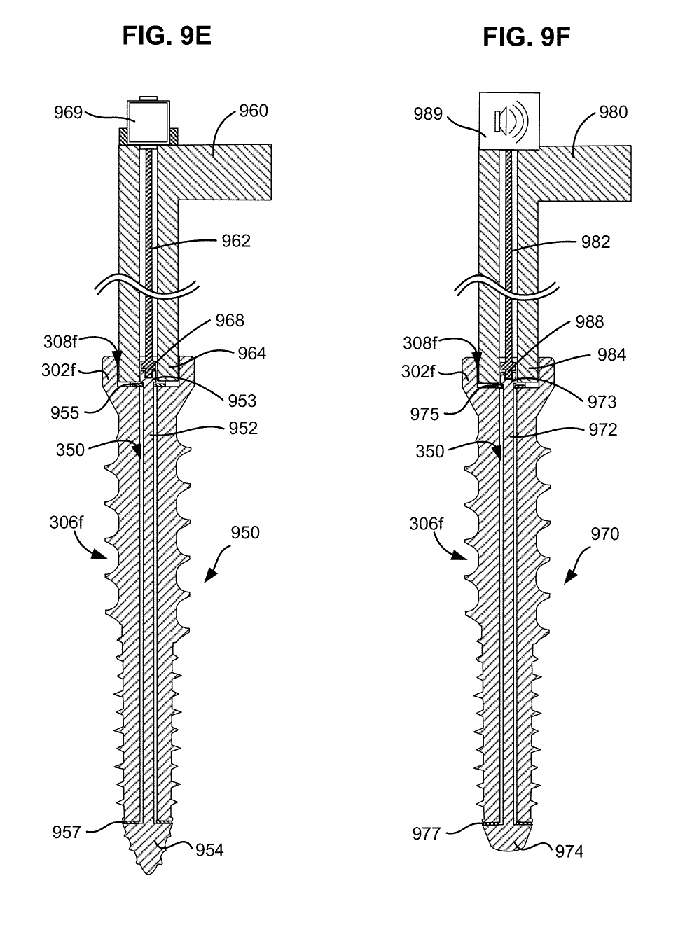

FIG. 9A illustrates a variation 910 of the cannulated bone anchor 300f previously described with respect to FIG. 3F in which the tip 304f is replaced and/or augmented with an integrated thermoelectric tip 914. A pair of conductors 912 (for example, insulated wires) pass through continuous bore 350 from thermoelectric tip 914 to head 302f. An electrical connector 916 provides for releasable connection of conductors 912 to a power supply 900. Power supply 900 is, thus, coupled to thermoelectric tip 914 via electrical connector 916 by conductors 912. The power supply 900 provides electrical energy to heat thermoelectric tip 914. Integrated thermoelectric tip 914 converts electrical energy into heat energy which is then transmitted by conduction into the bone cement to soften and/or melt the bone cement adjacent the tip of the bone anchor during implantation. For example, in one embodiment thermoelectric tip 914 includes one or more resistive heating elements. Power supply 900 drives an electrical current through the one or more resistive heating elements which generate heat in response. For example, in one embodiment, thermoelectric tip 914 includes one or more resistive heating elements. Power supply 900 drives an electrical current through the one or more resistive heating elements which generate heat in response. In an embodiment, the connection between conductors 912 and thermoelectric tip 914 are releasable such that the conductors 912 can be disconnected from thermoelectric tip 914 by pulling the proximal end of conductors 912 such that conductors 912 are removed from bone anchor 910 after implantation and are, therefore, not permanently implanted in the patient.

FIG. 9B illustrates a variation 920 of the cannulated bone anchor 300f previously described with respect to FIG. 3F in which the tip 304f is replaced and/or augmented with an integrated thermoelectric tip 924. Thermoelectric tip 924 can be blunt, tapered, or sharp, and can include the screw tip features previously disclosed including, but not limited to, one or more of threads, flutes, grooves, self tapping, drill, and a distal aperture. A single conductor 922 (for example, a titanium or stainless rod) passes through continuous bore 350 from thermoelectric tip 924 to head 302f. Conductor 922 may be spaced from shaft 306f by an air gap 928 to prevent short circuit. Alternatively, a sleeve made from an insulating biocompatible material (e.g. PEEK) is used to surround conductor 922. An electrical connector 926 provides for releasable connection of conductor 922 and shaft 306f to a power supply 900. Power supply 900 is, thus, coupled to thermoelectric tip 924 via electrical connector 916 through shaft 306f and conductor 922. The power supply 900 provides electrical energy to heat thermoelectric tip 924. Integrated thermoelectric tip 924 converts electrical energy into heat energy which is then transmitted by conduction into the bone cement to soften and/or melt the bone cement adjacent the tip 924 of the bone anchor 300f during implantation. For example, in one embodiment thermoelectric tip 924 includes one or more resistive heating elements. Power supply 900 drives an electrical current through the one or more resistive heating elements which generate heat in response. In an embodiment, the connection between conductor 922 and thermoelectric tip 924 is releasable such that the conductor 922 can be disconnected from thermoelectric tip 924 by pulling the proximal end of conductor 922 such that conductor 922 is removed from bone anchor 920 after implantation and is, therefore, not permanently implanted in the patient.

FIG. 9C illustrates a variation 930 of the polyaxial pedicle screw 660c previously described with respect to FIG. 6C in which the tip 104 is replaced and/or augmented with an integrated thermoelectric tip 934. One or more conductors 932 (for example, insulated wire(s)) pass through shaft 106 from thermoelectric tip 934 to a rotary electrical connector 936. Rotary electrical connector 936 provides for releasable connection of conductor(s) 932 to a power supply 900. Rotary electrical connector 936 is designed to rotate independent of shaft 106 while maintaining an electrical connection with conductor(s) 932 thereby allowing bone anchor 930 to be turned during implantation without interference from the connection to power supply 900. Power supply 900 is, thus, coupled to thermoelectric tip 934 via rotary electrical connector 936 through conductor(s) 932. The power supply 900 provides electrical energy to heat thermoelectric tip 934. Integrated thermoelectric tip 934 converts electrical energy into heat energy which is then transmitted by conduction into the bone cement to soften and/or melt the bone cement adjacent the tip of the bone anchor during implantation. For example, in one embodiment thermoelectric tip 934 includes one or more resistive heating elements. Power supply 900 drives an electrical current through the one or more resistive heating elements which generate heat in response. In an embodiment, the connection between rotary electrical connector 936 and shaft 106 is releasable such that the rotary electrical connector 936 can be disconnected from shaft 106 after implantation.

FIG. 9D illustrates a variation 940 of the cannulated bone anchor 300f previously described with respect to FIG. 3F in which the tip 304f has no tip aperture but is augmented with an integrated thermoelectric element 944. Tip 304f can be blunt, tapered, or sharp, and can include the screw tip features previously disclosed including, but not limited to, one or more of threads, flutes, grooves, self tapping, drill, and a distal aperture. A single conductor 942 (for example, a titanium rod, a stainless rod, or a copper wire) passes through continuous bore 350 from thermoelectric element 944 to head 302f. Conductor 942 may be spaced from shaft 306f by an air gap 948 to prevent short circuit. Alternatively, a sleeve made from an insulating biocompatible material (e.g. PEEK) is used to surround conductor 942. An electrical connector 946 provides for releasable connection of conductor 942 and shaft 306f to a power supply 900. Power supply 900 is, thus, coupled to thermoelectric element 944 via electrical connector 946 through shaft 306f and conductor 942. The power supply 900 provides electrical energy to heat thermoelectric element 944 which thereby heats tip 304f. Thermoelectric element 944 converts electrical energy into heat energy which is then transmitted by conduction through tip 304f into bone cement to soften and/or melt the bone cement adjacent the tip of the bone anchor during implantation. For example, in one embodiment, thermoelectric element 944 is a high resistivity material, for example, Nichrome 80/20, Kanthal, Cupronickel alloy, Molybedenum disilicide, or PTC ceramic. Power supply 900 drives an electrical current through the high resistivity material which generates heat in response. In an embodiment, the connection between conductor 942 and thermoelectric element 944 is releasable such that the conductor 942 can be disconnected from thermoelectric element 944 by pulling the proximal end of conductor 942 such that conductor 942 is removed from bone anchor 940 after implantation and is, therefore, not permanently implanted in the patient.

In using a bone anchor having a thermoelectric tip as disclosed, for example, in FIGS. 9A-9D, the physician connects the power supply to the electrical connector of the bone anchor. The physician then operates the power supply 900 to raise the temperature of the thermoelectric tip to a temperature suitable for softening and/or melting bone cement. The physician utilizes a wrench to drive the bone anchor into the bone cement while the thermoelectric tip is maintained at the desired temperature. The thermoelectric tip heats the bone cement adjacent the thermoelectric tip. Melted/softened bone cement flows away from the thermoelectric tip as the thermoelectric tip is driven into the bone thereby creating a bore simultaneous with implantation. The thermoelectric tip and/or shaft of the bone anchor are, in some embodiments, provided with channels and/or grooves which allow softened/melted bone cement to flow away from the thermoelectric tip during implantation of the bone anchor. When the bone anchor has been implanted to its desired position in the bone, the power supply 900 is disconnected from the electrical connector.

Power supply 900 can be a conventional surgical power supply commonly available in an operating room, for example, a bovie or cautery power supply. However, in a preferred embodiment, the temperature of the thermoelectric tip is monitored and regulated by power supply 900 such that thermoelectric tip achieves, and remains at a temperature suitable for softening and/or melting bone cement during implantation of the bone anchor without damaging surrounding tissues or burning the bone cement. For example, in the thermoelectric tip can include one or more resistive heating elements. Power supply 900 drives an electrical current through the one or more resistive heating elements which generate heat in response. Power supply 900 can preferably monitor the resistance of the resistive heating elements in order to assess the temperature of the thermoelectric tip and modulate the supplied current in order to achieve and regulate a desired temperature of the thermoelectric tip. The temperature necessary for melting bone cement is variable dependent upon the composition of the bone cement. Thus, in some embodiments, the power supply 900 includes a control for selecting the temperature to which the thermoelectric tip is raised—for example, between 100° C. and 200° C.

FIG. 9E illustrates a variation 950 of the cannulated bone anchor 300f previously described with respect to FIG. 3F in which the tip 304f is replaced with an integrated burr tip 954. Burr tip 954 can be blunt, tapered, or sharp, and can include the screw tip features previously disclosed including, but not limited to, one or more of threads, flutes, grooves, self tapping, drill, and a distal aperture. A shaft 952 (for example, a titanium rod or stainless steel rod) passes through continuous bore 350 from burr tip 954 to head 302f. The proximal end of shaft 952 includes a mechanical power coupling 953 (for example, a square or hex socket or shaft end). Shaft 952 can be formed in one piece with burr tip 954 from titanium. A snap-ring/bushing 955 secures burr tip 954 and shaft 952 within bone anchor 300f and/or reduces the friction between coupling 953 and head 302f. Another bushing 957 optionally reduces friction between the distal end of shaft 306f and burr tip 954. Burr tip 954, shaft 952 and coupling 953 rotate as one unit and can rotate independently of shaft 306f.

During implantation, the physician utilizes a wrench 960 which has a head 964 adapted to engage socket 308f of bone anchor 300f in order to turn bone anchor 300f. Wrench 960 includes a motor 969 coupled to drive shaft 962 which has at its distal end mechanical power coupling 968 designed to engage the mechanical power coupling 953 of bone anchor 950. When engaged motor 969 can be operated to rotate the burr tip 954 at high speed, friction between burr tip 954 and bone cement adjacent burr tip 954 raises the temperature of burr tip 954 and the bone cement softening and/or melting the bone cement. The burr tip 954 advances through the bone cement as the physician utilizes wrench 960 to rotate bone anchor 950 independent of the rotation of burr tip 954. The bone cement flows away from burr tip 954 as burr tip 954 is introduced, creating the distal bore simultaneous with implantation. Bone anchor 950 is, in some embodiments, provided with channels and/or grooves which allow melted bone cement to flow away from burr tip 954. When the bone anchor has been implanted in the desired position, wrench 960 is removed. In this procedure burr tip 954 is used to soften and/or melt the bone cement during implantation of the bone anchor thereby reducing the possibility of fracture.

FIG. 9F illustrates a variation 970 of the cannulated bone anchor 300f previously described with respect to FIG. 3F in which the tip 304f is replaced with an integrated ultrasound tip 974. Ultrasound tip 974 can be blunt, tapered, or sharp, and can include the screw tip features previously disclosed including, but not limited to, one or more of threads, flutes, grooves, and a distal aperture. A shaft 972 (for example, a titanium rod or stainless steel rod) passes through continuous bore 350 from ultrasound tip 974 to head 302f. The proximal end of shaft 972 includes an ultrasound coupling 973, for example, a socket or shaft end. Shaft 972 can be formed in one piece with ultrasound tip 974 from titanium. A snap-ring/bushing 975 secures ultrasound tip 974 and shaft 972 within bone anchor 300f and/or vibrationally isolates coupling 973 from head 302f. Another bushing 977 optionally vibrationally isolates the distal end of shaft 306f and ultrasound tip 974. Ultrasound tip 974, shaft 972 and coupling 973 can vibrate ultrasonically independent of vibration of shaft 306f.

During implantation, the physician utilizes a wrench 980 which has a head 984 adapted to engage socket 308f of bone anchor 300f in order to turn bone anchor 300f. Wrench 980 includes an ultrasound transducer 989 coupled to shaft 982 which has at its distal end an ultrasound coupling 988 designed to engage the ultrasound coupling 973 of bone anchor 970. When engaged, ultrasound transducer 989 can be operated to send ultrasound vibrations to ultrasound tip 974 via shaft 982. (In an alternative embodiment, ultrasound frequency vibrations are induced directly in ultrasound coupling 973 by a device located in the head 984 of wrench 980.) Friction caused by high frequency vibration between ultrasound tip 974 and bone cement adjacent ultrasound tip 974 raises the temperature of ultrasound tip 974 and/or the bone cement softening and/or melting the bone cement. The ultrasound tip 974 advances through the bone cement as the physician utilizes wrench 980 to rotate bone anchor 970. The bone cement flows away from ultrasound tip 974 as ultrasound tip 974 is introduced, creating the distal bore simultaneous with implantation. Bone anchor 970 is, in some embodiments, provided with channels and/or grooves which allow melted bone cement to flow away from ultrasound tip 974. When the bone anchor has been implanted in the desired position, wrench 980 is removed. In this procedure, ultrasound tip 974 is used to soften and/or melt the bone cement during implantation of the bone anchor thereby reducing the possibility of fracture of the bone cement.

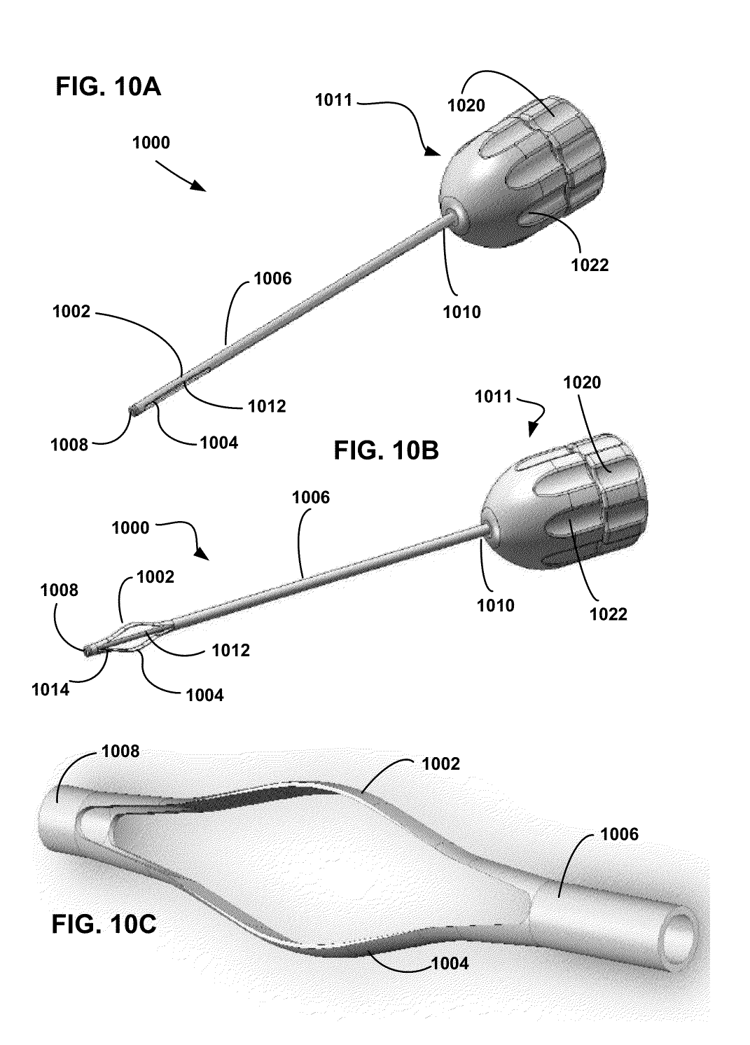

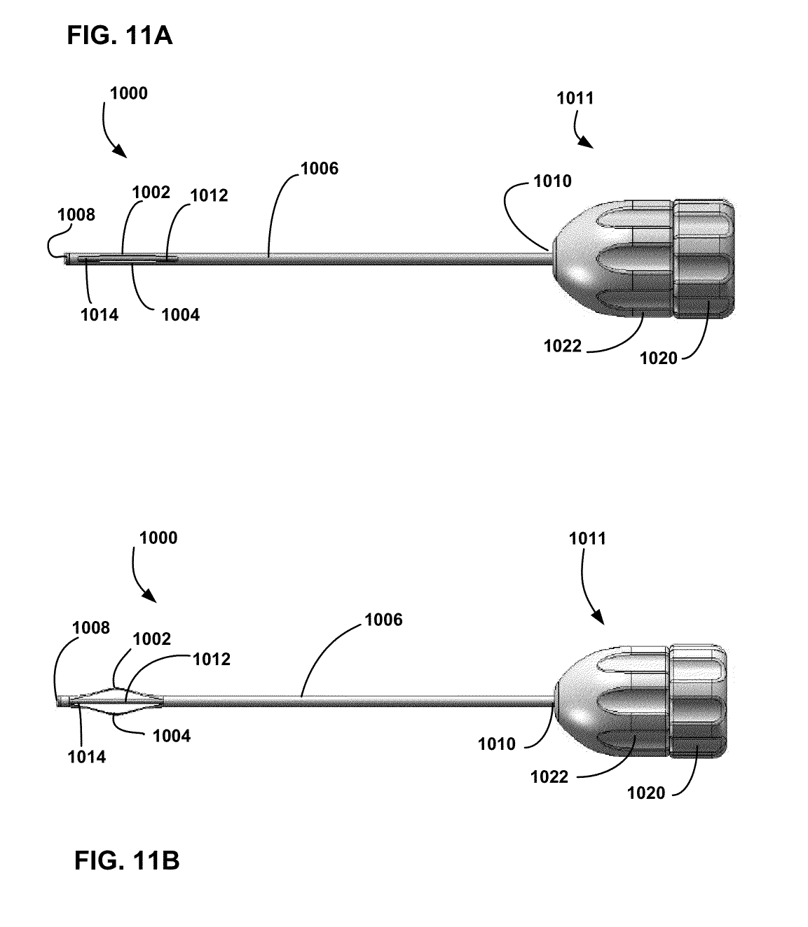

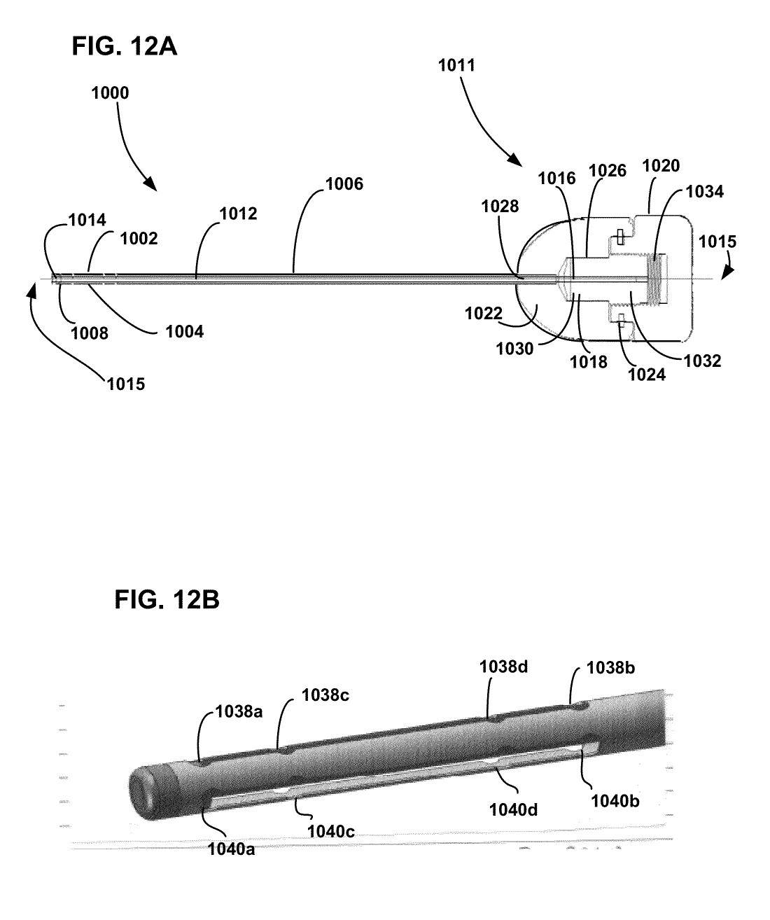

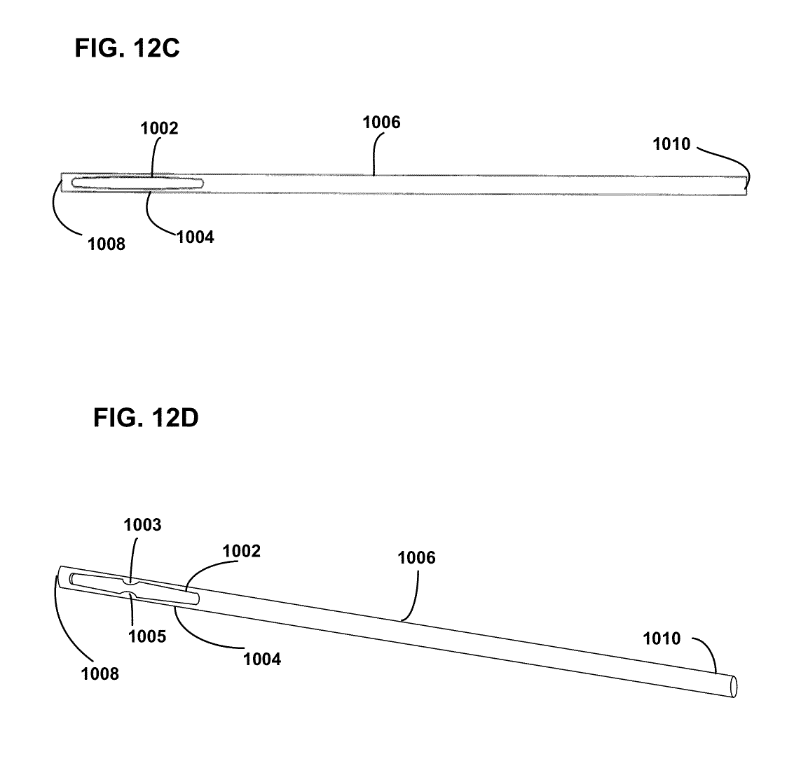

FIGS. 10A and 10B depict perspective views of an embodiment of the bone cutting tool 1000 of the invention with the first cutting blade 1002 and the second cutting blade 1004 in the non-expanded and expanded positions, respectively. Further, FIGS. 11A, 11B and 12C depict side views of an embodiment of the bone cutting tool 1000 of the invention with the first cutting blade 1002 and the second cutting blade 1004 in the non-expanded and expanded positions, respectively. It is to be understood that an alternative embodiment of the invention can include a single cutting blade that works, for example, like the first cutting blade or the second cutting blade. For all the embodiments described herein, the edges of the blades, such as blades 1002 and 1004 can be sharpened or tapered in order to enhance the cutting ability of the blades.

The first and second blades are formed in an outer tube 1006 which has a distal end 1008 and a proximal end 1010. As the first and second cutting blade 1002, 1004 are formed from a tube, in a preferred embodiment, in a plane perpendicular to and over the longitudinal axis, the blades are curved. The proximal end 1010 of the outer tube 1006 is secured to a handle 1011. An inner rod 1012 is positioned in the outer tube 1006. The inner rod 1012 includes a distal end 1014 which is secured to the distal end 1008 of the outer tube 1006 and a proximal end 1016 (shown in FIG. 12A) which is secured relative to the proximal end of the outer tube 1002 to the handle 1011. The inner rod 1012 also has a longitudinal axis 1015 which serves additionally as the longitudinal axis of the tool 1000. The outer tube and the inner rod can be stainless steel, or a superelastic material such Nitinol (Niti), or titanium. Alternatively, the tube and cutting blades can be made of a superelastic material and the inner rod can be made of stainless steel or titanium. In a preferred embodiment of the invention, the cutting blades are made of a superelastic material such as Nitinol so that the cutting blades can flexibly expand and contract.

As can be seen, for example, in the combination of FIGS. 10A, 10B, 11A, 11B and FIG. 12A, the handle 1011 includes a first part 1018 which is secured to the proximal end 1016 of the inner rod 1012. The handle 1011 also includes a second part 1020 and a third part 1022. The second part 1020 of the handle 1011 is secured to the third part 1022 of the handle 1011 by a ring 1024 that fits into grooves shown in the second part 1020 and the third part 1022 of the handle 1011. Further, the second part 1020 of the handle 1011 can rotate relative to the third part 1022 of the handle 1011 due to the ring 1024. As indicated above, the first part 1018 of the handle 1011 is secured to said proximal end 1016 of the inner rod 1012. The third part 1022 of the handle 1011 includes a first bore 1026 (shown in FIG. 12) which slidingly receives a distal end 1030 of the first part 1018 of the handle 1011 and a second bore 1028 which receives the proximal end of the inner rod 1012. The first bore 1026 communicates with the second bore 1028. The second part 1020 of the handle 1011 includes a threaded third bore 1034 which receives a threaded proximal end 1032 of the first part 1018 of the handle 1011.

Accordingly, rotation of the second part 1020 of the handle 1011 relative to the third part 1022 of the handle 1011 causes the first part 1018 of the handle 1011 to move causing the rod 1012 to move relative to the outer tube 1006. Rotation of the second part 1020 of the handle 1011 in one direction causes the inner rod to move distally relative to the outer tube and rotation of the second part of the handle in the opposite direction causes the inner rod to move proximally relative to the outer tube.

As can be seen in FIGS. 10B and 11B, movement of the inner rod in a proximal direction towards the handle 1011 causes the first blade 1002 and the second blade 1004, respectively, to move to an expanded configuration. As depicted in FIGS. 10A and 11A movement of the inner rod distally relative to the outer tube causes the first and second cutting blades to contrast to the original shape of the tube.

In a preferred embodiment of the invention, the cutting blades are made of a superelastic material such as Nitinol so that the cutting blades can flexibly expand and contract.

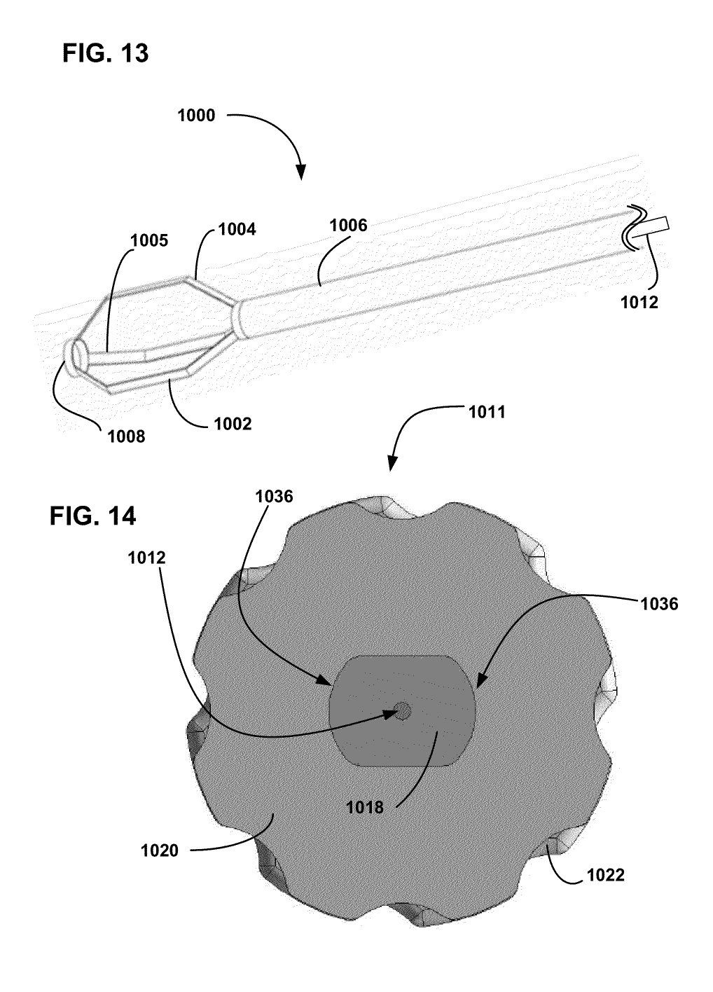

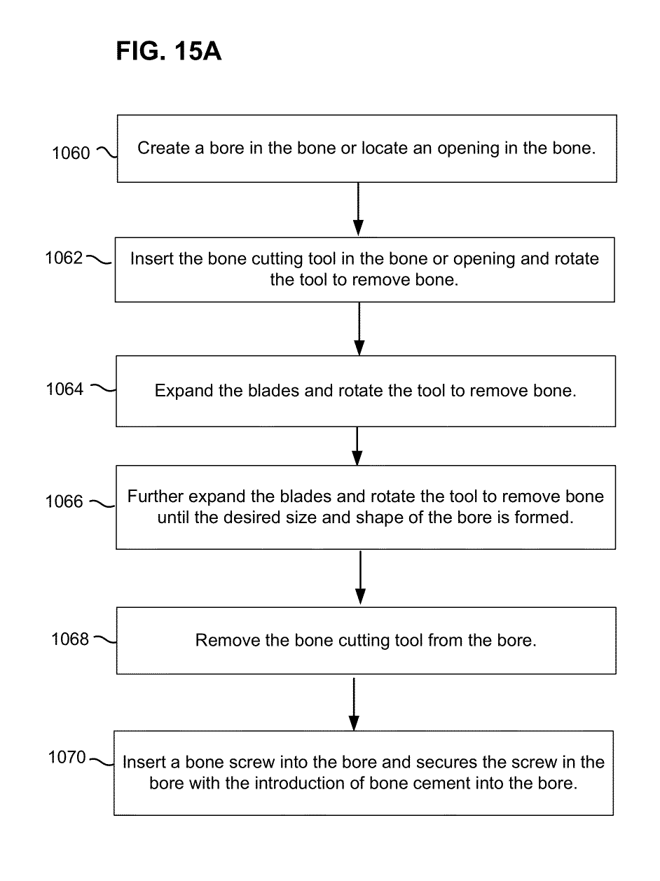

As can be seen in FIG. 14, the first part 1018 of the handle 1011 includes “D” shaped ends 1036 that fit into a corresponding shaped first bore 1026 of FIG. 12A of the third part 1022 of the handle 1011 to prevent the first part 1018 and the inner rod 1002 from rotating when the second part of the handle rotates relative to the first part of the handle 1011. Other features such as the use of a single “D” shaped end and a longitudinal key (not shown) could prevent the rod from rotating relative to the outer tube.

As can be seen in FIGS. 12A, 12B and 13, the first and second cutting blades 1002, 1004 include recesses or weakened portions or sections that promote bending of one portion of each of the first and second cutting blades relative to another portion of the respective first and second cutting blades. First cutting blade 1002 includes end recesses 1038a and 1038b as well as middle recesses 1038c and 1038d. Second cutting blade 1004 includes end recesses 1040a and 1040b as well as middle recesses 1040c and 1040d. Due to these recesses and as seen in FIG. 13, when the inner rod 1012 is moved relative to the outer tube 1006 in order to expand the cutting blades 1002 and 1004, the portion of the first cutting blade 1002 between middle recesses, 1038c and 1038d and the portion of the second cutting blade 1004 between middle recesses 1040c and 1040d expand substantially in a manner to remain parallel to the longitudinal axis of the inner rod 1012. Thus, the cutting blades take on a cylindrical shape in order to cut a cylindrical bore in the bone. This is in contrast to the more curved or somewhat parabolic shaping that the expanded bone cutting blades can take in other embodiments of the invention as shown in FIGS. 10B and 11B where, by way of example only, the blades are made of superelastic material. As can be seen in FIGS. 12D and 13, a third bone cutting blade 1005 can be formed in the outer tube 1006. In a preferred embodiment, the blades are formed in the tube of superelastic material such as Nitinol using laser cutting techniques.

In an alternative embodiment, the unexpanded first and second cutting blade 1002, 1004 in FIG. 12D have a modified structure, with a broader, wider and/or flatter middle portion 1003, 1005 cut into the outer tube. The geometry of this cut influences the expanded shape of the first and second cutting blade 1002, 1004. With a broader or wider or flatter middle section, the middle section tends to stay more flat and parallel to the longitudinal axis, than do the parabolic shaped expanded blades 1002, 1004 of FIG. 10C.

In yet another alternative embodiment, the cutting blades can be spiral in shape and also have teeth cut into the edge of the blades or the edges of the blades can be serrated.

In the embodiments of the invention, it is to be understood that the outer tube with the one or more cutting blades and the inner rod can be selectively connected to the handle so that the outer tube and the inner rod can be replaceable with the reusable handle. A release mechanism for selectively connecting the outer tube with the one or more cutting blades and the inner rod to the handle are well known in the art.

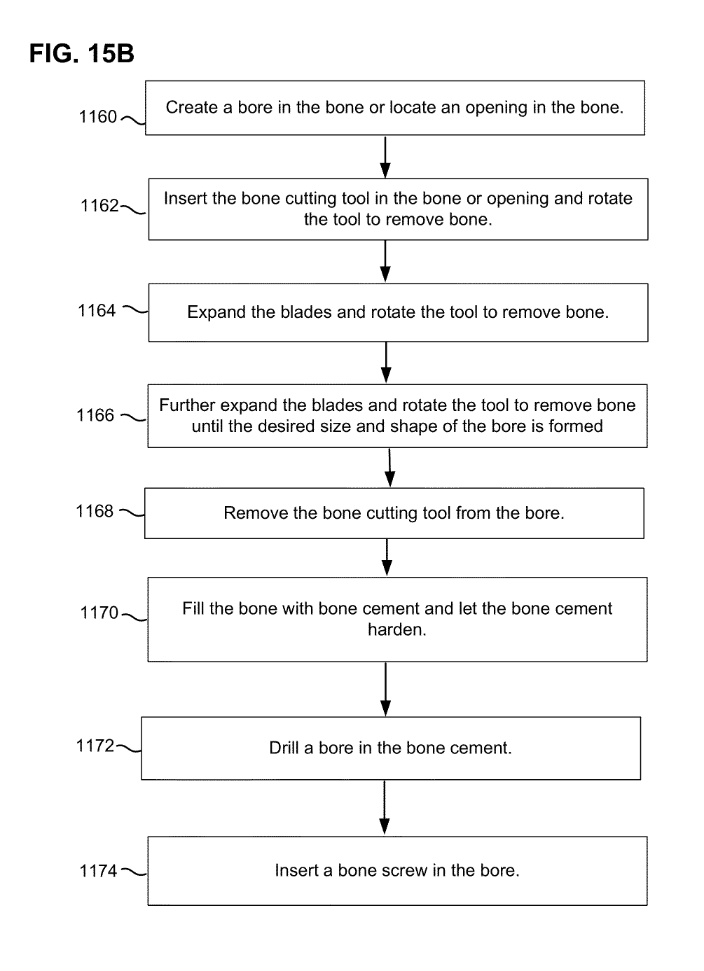

As can be seen in FIG. 15A, an embodiment of the method of the invention includes the following steps. At step 1060, a bore is created in the bone or an opening is identified in the bone. At step 1062, the bone cutting tool 1000 is inserted into the bore or the identified opening. The tool may be rotated to remove or cut away bone. At step 1064, the first and second blades are expanded and the tool is further rotated to remove bone. At step 1066, the first and second blades are further expanded and rotated and this is continued until the bore in the bone achieves the desired size. At step 1068, the bone cutting tool is removed from the bore. Such a removal step may require the cutting blades to be contracted using the handle 1011. At step 1070, a bone screw is introduced into the bore and either one or both of bone cement is introduced into the bore between the bore and the bone screw and/or the bone cement is introduced through channels, bores and ports formed in the screw (see FIG. 3F) and into the bore. The bone cement is allowed to flow into the porous bone to dry, thereby securing the bone screw to the bone. It is to be understood that in practice, the bone screw will have threads thereof engage some portions of the bore, but not other portions, as the bore is formed in porous bone. Thus, the bone cement will ensure that the voids in the porous bone are filled and that the thread of the bone screw will engage the bone cement if bone is not available.

As can be seen in FIG. 15B, an embodiment of the method of the invention includes the following steps. At step 1160, a bore is created in the bone or an opening is identified in the bone. At step 1162, the bone cutting tool 1000 is inserted into the bore or the identified opening. The tool may be rotated to remove or cut away bone. At step 1164, the first and second blades are expanded and the tool is further rotated to remove bone. At step 1166, the first and second blades are further expanded and rotated and this is continued until the bore in the bone achieves the desired size. At step 1168, the bone cutting tool is removed from the bore. Such a removal step may require the cutting blades to be contracted using the handle 1011. At step 1170, the bore is filled with bone cement and the bone cement is allowed to dry. At step 1172, a bore is drilled or created in the dried bone cement. At step 1174, a bone screw is inserted into the bore in the bone cement.

It is also to be understood that the system and method of embodiments of the invention can be used to create and expand bores in the other tissue of the body in addition to creating and expanding bores in the bone of the vertebral body. For example, the system and method of embodiments of the invention can be used to create and expand bores in the disks that are located between the vertebral bodies of the spine. Further embodiments of the inventions can be used to create and expand bores in other soft tissue and bone of the body.

### Materials

The bone anchor, implantation tools, deflectable post, spinal rods, spinal plates, and other spinal implant components are preferably made of biocompatible and/or implantable metals. The bone anchor and implantation tools can, for example, be made of titanium, titanium alloy, cobalt chrome alloy, a shape memory metal, for example, Nitinol (NiTi) or stainless steel. In preferred embodiments, the bone anchor is made of titanium alloy; however, other materials, for example, stainless steel may be used instead of, or in addition to, the titanium\titanium alloy components. Typically, the tip, proximal shaft, distal shaft, and head (or at least that portion of the head attached to the proximal shaft) are formed in one piece from titanium\titanium alloy\stainless steel. The bone anchor may be cast and/or molded in one piece and/or machined from a block of metal using methods known in the art. In alternative embodiments one or more elements of the bone anchor are formed separately and then joined to the other components during manufacturing.

The foregoing description of preferred embodiments of the present invention has been provided for the purposes of illustration and description. It is not intended to be exhaustive or to limit the invention to the precise forms disclosed. Many embodiments were chosen and described in order to best explain the principles of the invention and its practical application, thereby enabling others skilled in the art to understand the invention for various embodiments and with various modifications that are suited to the particular use contemplated.

The particular bone anchor embodiments shown herein are provided by way of example only. The bone anchors have been described with particular reference to spinal stabilization, however, the invention disclosed herein and bone anchors embodying it may find application in any bone or orthopedic application where a bone anchor/bone screw is desired to be secured in a bone which includes hardened bone cement. It is an aspect of preferred embodiments of the present invention that a range of bone anchors are provided (for example, in a kit) and that different of the bone anchors have different combinations of the shafts, tips, heads and other features disclosed herein. Particular bone anchors may incorporate any combination of the shafts, tips, heads and other features disclosed herein, and in the application incorporated by reference, and standard spinal stabilization and/or fusion components, for example, screws, pedicle screws, polyaxial screws and rods. Additionally, any of the implantation tools and methods described herein and in the related application incorporated by reference can be used or modified for use with such bone anchors. It is intended that the scope of the invention be defined by the claims and their equivalents.

1. A method for creating a bore in a vertebra comprising the steps of:
inserting a bone cutting tool with a longitudinal axis and at least one cutting blade into a vertebra;
turning the bone cutting tool in order to cut bone in the vertebra and expand a bore within which the cutting tool is positioned;
expanding the cutting blade away from the longitudinal axis;
turning the bone cutting tool with the expanded cutting blade to cut additional bone in the vertebra and further expand the bore in vertebra; and
as necessary, continuing to expand the cutting blade away from the longitudinal axis and turning the cutting tool in the bore in the vertebra until the bore is the desired size.

2. The method of claim 1 including the step of:
inserting bone cement and a bone screw in the bore; and
fixing the position of the bone screw in the bore by allowing the bone cement to dry.

3. The method of claim 1 including the step of:
inserting a bone screw into the bore, which bone screw has channels through which bone cement can be injected; and
injecting the bone cement through the channels in the bone screw and into the bore in the vertebra in order to fix the position of the bone screw in the vertebra.

4. The method of claim 1 including the step of:
drilling a bore in the vertebra prior to inserting the bone cutting tool into the vertebra.

5. The method of claim 1 including the step of:
wherein the bone cutting tool includes a rod positioned along the longitudinal axis and tube extending over the rod;
wherein said tube at a distal end includes the at least one cutting blade and a second cutting blade; and
wherein said expanding step includes moving said rod relative to said tube in order to expand the at least one cutting blade and the second cutting blade relative to said rod.

6. The method of claim 5 wherein said expanding step includes retracting said rod in order to expand the at least one cutting blade and the second cutting blade.

7. The method of claim 5 wherein said expanding step includes moving said rod along the longitudinal axis without rotating said rod.

8. The method of claim 1 wherein said at least one blade is made of a superelastic material and said expanding step includes flexibly expanding said at least one blade.

9. The method of claim 5 wherein said at least one cutting blade and said second cutting blade are spaced from each other by slots; and
said expanding step includes moving said at least one cutting blade apart from said second cutting blade by expanding the slots.

10. The method of claim 5 wherein said at least one cutting blade and said second cutting blade include weakened sections such said expanding step includes expanding said cutting blades such that said cutting blades cut a cylindrical bore in the vertebra.

11. The method of claim 1 wherein said bone cutting tool includes a handle with a first portion and a second portion; and
wherein said expanding step includes moving the first portion of said handle relative to the second portion of the handle.

12. The method of claim 5 wherein said bone cutting tool includes a handle with a first portion and a second portion; and
wherein said expanding step includes moving said first portion relative to said second portion such that said cutting blades move relative to said rod.

13. The method of claim 1 including the step of:
contracting the bone cutting blade in order to remove the bone cutting tool from the bore.

14. A method for creating a bore in a vertebra comprising the steps of:
inserting a bone cutting tool with a longitudinal axis and a first cutting blade and a second cutting blade into a vertebra;
turning the bone cutting tool in order to cut bone in the vertebra and expand a bore within which the cutting tool is positioned;
expanding the cutting blade away from the longitudinal axis;
wherein the first and second blade are made of a superelastic material and said expanding step includes flexibly expanding said first and second blades;
turning the bone cutting tool with the expanded cutting blades to cut additional bone in the vertebra and further expand the bore in vertebra;
as necessary, continuing to expand the cutting blades away from the longitudinal axis and turning the cutting tool in the bore in the vertebra until the bore is the desired size;
wherein the bone cutting tool includes a rod positioned along the longitudinal axis and tube extending over the rod;
wherein said tube at a distal end includes the first cutting blade and second cutting blade; and
wherein said expanding step includes moving said rod relative to said tube in order to expand the first cutting blade and the second cutting blade relative to said rod.

15. The method of claim 14 including the step of:
inserting bone cement and a bone screw in the bore; and
fixing the position of the bone screw in the bore by allowing the bone cement to dry.

16. The method of claim 14 including the step of:
inserting a bone screw into the bore, which bone screw has channels through which bone cement can be injected; and
injecting the bone cement through the channels in the bone screw and into the bore in the vertebra in order to fix the position of the bone screw in the vertebra.

17. The method of claim 14 including the step of:
drilling a bore in the vertebra prior to inserting the bone cutting tool into the vertebra.

18. The method of claim 14 wherein said expanding step includes retracting said rod in order to expand the first cutting blade and the second cutting blade.

19. The method of claim 14 wherein said expanding step includes moving said rod along the longitudinal axis without rotating said rod.

20. The method of claim 14 wherein said first cutting blade and said second cutting blade are spaced from each other by slots; and
said expanding step includes moving said first cutting blade apart from said second cutting blade by expanding the slots.

21. The method of 14 wherein said first cutting blade and said second cutting blade include weakened sections such said expanding step includes expanding said cutting blades such that said cutting blades cut a cylindrical bore in the vertebra.

22. The method of claim 14 wherein said bone cutting tool includes a handle with a first portion and a second portion; and
wherein said expanding step includes moving the first portion of said handle relative to the second portion of the handle.

23. The method of claim 14 wherein said bone cutting tool includes a handle with a first portion and a second portion; and
wherein said expanding step includes moving said first portion relative to said second portion such that said cutting blades move relative to said rod.

24. The method of claim 14 including the step of:
contracting the bone cutting blade in order to remove the bone cutting tool from the bore.

25. A method for creating a bore in a vertebra comprising the steps of:
inserting a bone cutting tool with a longitudinal axis and a first cutting blade and a second cutting blade into a vertebra;
turning the bone cutting tool in order to cut bone in the vertebra and expand a bore within which the cutting tool is positioned;
expanding the cutting blade away from the longitudinal axis;
turning the bone cutting tool with the expanded cutting blades to cut additional bone in the vertebra and further expand the bore in vertebra;
as necessary, continuing to expand the cutting blades away from the longitudinal axis and turning the cutting tool in the bore in the vertebra until the bore is the desired size;
wherein the bone cutting tool includes a rod positioned along the longitudinal axis and tube extending over the rod;
wherein said tube at a distal end includes the first cutting blade and second cutting blade; and
wherein said expanding step includes moving said rod relative to said tube in order to expand the first cutting blade and the second cutting blade relative to said rod;
wherein said bone cutting tool includes a handle with a first portion and a second portion; and
wherein said expanding step includes moving said first portion relative to said second portion such that said cutting blades move relative to said rod.

26. The method of claim 1 wherein the at least one cutting blade includes weakened sections in order to influence a shape of the blade as the blade is expanded during the expanding step.

27. The method of claim 1 wherein the at least one cutting blade includes an expanded section in order to influence a shape of the blade as the blade is expanded during the expanding step.

28. The method of claim 14 wherein the first and second cutting blades include weakened sections in order to influence a shape of the blades as the blades are expanded during the expanding step.

29. The method of claim 14 wherein the first and second cutting blades include an expanded section in order to influence a shape of the blades as they are expanded during the expanding step.

30. The method of claim 1 wherein the at least one cutting blade includes an unexpanded shape in order to influence an expanded shape of the blade as the blade is expanded during the expanding step.

31. The method of claim 1 including the steps of:
filling the bore with bone cement and allowing the bone cement to dry;
creating a bore in the bone cement; and
inserting a bone screw in the bore created in the bone cement.

32. The method of claim 14 including the steps of:
filling the bore with bone cement and allowing the bone cement to dry;
creating a bore in the bone cement; and
inserting a bone screw in the bore created in the bone cement.

33. A method for creating a bore in tissue of a body of a patient comprising the steps of:
inserting a tissue cutting tool with a longitudinal axis and at least one cutting blade into tissue of the body;
turning the tissue cutting tool in order to cut tissue in the body and expand a bore within which the cutting tool is positioned;
expanding the cutting blade away from the longitudinal axis;
turning the tissue cutting tool with the expanded cutting blade to cut additional tissue in the body and further expand the bore in body; and
as necessary, continuing to expand the cutting blade away from the longitudinal axis and turning the cutting tool in the bore in the body until the bore is the desired size.
